SEQUENCE LISTING (7 sequences)

SEQ ID NO: 1 - protein, 13 aa, artificial sequence
  synthetic protein
ICVVQDWGHHRCT

SEQ ID NO: 2 - protein, 6 aa, artificial sequence
  synthetic protein
  MISC_FEATURE (1)..(1): acetyl phenylalanine
  MISC_FEATURE (2)..(2): L-ornithine
  MISC_FEATURE (4)..(4): D-cyclohexylalanine
XXPXWR

SEQ ID NO: 3 - protein, 10 aa, artificial sequence
  synthetic protein
IRCPRPQDFE

SEQ ID NO: 4 - protein, 5 aa, Homo sapiens
SGTPS

SEQ ID NO: 5 - protein, 6 aa, Homo sapiens
DGETAV

SEQ ID NO: 6 - protein, 7 aa, Homo sapiens
TPQEVEA

SEQ ID NO: 7 - protein, 7 aa, Homo sapiens
FPEDVAP

(19) United States
(12) Patent Application Publication
(10) Pub. No.: US 2007/0123466 A1
(43) Pub. Date: May 31, 2007

(54) Method of treating recurrent miscarriages

(75) Inventors: Jane E. Salmon, New York, NY (US); Guillermina Girardi, New York, NY (US); V. M. Holers, Denver, CO (US)

Correspondence Address: DARBY & DARBY P.C., P. O. BOX 5257, NEW YORK, NY, 10150-5257, US

(73) Assignees: New York Society for the Ruptured and Crippled Maintaining The Hospital for Special Surgery, New York, NY (US); The Regents of the Universuty of Colorado, Boulder, CO (US)

(21) Appl. No.: 10/556,493

(22) PCT Filed: May 13, 2004
(86) PCT No.: PCT/US04/15040

§ 371 Date: Dec. 22, 2005

Related U.S. Application Data
(60) Provisional application No. 60/470,444, filed on May 13, 2003.

(51) Int. Cl. A61K 38/08; A61K 38/10

(52) U.S. Cl. 514/14; 514/15; 514/16; 514/17

(57) ABSTRACT

Described are methods for treating, preventing, or reducing the risk of, miscarriages, especially recurrent miscarriages. The methods comprise administering to a female subject a therapeutic agent that modulates the activity or binding of components of the complement system, together with a pharmaceutically acceptable carrier. For example, the therapeutic agent can be a C3-convertase inhibitor, an antibody against C5, an antagonist of the C5a receptor, or an antibody against factor B or factor D. Screening methods for agents that can prevent or reduce the risk of miscarriages, especially recurrent miscarriages, are also described.

### CROSS-REFERENCE TO RELATED APPLICATIONS

This Application is a Non-Provisional of Provisional (35 USC 119(e)) application 60/470,444 filed on May 13, 2003.

### STATEMENT REGARDING FEDERALLY SPONSORED RESEARCH OR DEVELOPMENT

The experiments performed in this application were supported in part by the National Institutes of Health, Grant Nos. AI-31105, AI25011, and GM-62134. The U.S. Government has a paid-up license in this invention and the right in limited circumstances to require the patent owner to license others on reasonable terms as provided for by the terms of these grants.

### FIELD OF THE INVENTION

This invention relates to methods of preventing or reducing the risk of miscarriages, particularly recurrent miscarriages.

### BACKGROUND OF THE INVENTION

The journey from conception to birth is fraught with danger. It has been estimated that 50% to 70% of all conceptions fail. Complications that occur during pregnancy remain a serious clinical problem and the triggers and mediators of placental and fetal damage are poorly understood. Recurrent pregnancy loss affects 1% to 3% of couples. In addition, preterm birth occurs in up to 10% of pregnancies, accounting for 70% of neonatal deaths and related neonatal morbidity, including neurological, respiratory, and metabolic complications in the newborn. The cost of caring for such conditions has been estimated at 5 to 6 billion dollars annually. Despite aggressive attempts to understand the basic biology underlying neonatal death and morbidity, their incidence has remained unchanged over the past 20 to 30 years. Furthermore, because in 50% to 60% of cases the well-established genetic, anatomic, endocrine, and infectious causes of fetal damage are not demonstrable, further work is necessary to elucidate the etiology of these complications of pregnancy.

Up to 2% of women in the U.S. suffer recurrent miscarriages. Though the cause of recurrent miscarriages in most women is unknown, an immune mechanism, involving the inappropriate and subsequently injurious recognition of the conceptus (i.e., the product of conception, including embryo and membranes) by the mother's immune system, has been proposed (American College of Obstetricians and Gynecologists, ACOG Practice Bulletin #24, ACOG, Washington, D.C., 2001; Clark et al., Hum Reprod Update 2001;7:501-511; and Mellor et al., Nat Immunol 2001;2:64-8). For example, Mellor et al. (2001), using an animal model, found that indoleamine-2,3-dioxygenase activity during pregnancy protected the fetus from a maternal immune response caused by paternally inherited antigens. Some clinics therefore promote “immune modulatory” treatments such as, e.g., the administration of intravenous IgG, or lymphocytes isolated from the prospective father, to treat recurrent miscarriages. These treatments, aimed at altering the incipient immune interaction between mother and conceptus by pre-conception treatments, have only met with limited success (Coulam et al., Am J Reprod Immunol 1995;34:333-338; Mowbray et al., Lancet 1985;1:941-943; Stephenson et al., Am J Reprod Immunol 1998;39:82-88) and are currently not endorsed by either the American College of Obstetricians and Gynecologists or the Royal College of Obstetricians and Gynecologists.

In a particular disorder, termed the antiphospholipid syndrome (APS), recurrent miscarriages are caused by the immune system's own production of anti-phospholipid antibodies. Patients with systemic lupus erythematosus (SLE) are particularly prone to APS, but individuals with other autoimmune features may also have anti-phospholipid antibodies. An in vivo study in an animal model of APS indicated that activation of one of the components of the complement system, C3, was required for the anti-phospholipid antibody-induced fetal loss in this model (Holers et al., J Exp Med 2002;195:211-20), and the administration of a recombinant version of a C3-convertase inhibitor, complement receptor 1-related gene/protein y (Crry) reduced fetal loss in the APS model. Another study demonstrated that the Crry protein is necessary for mouse embryos to survive (Xu et al., Science 2000;287:498-501; comment in Science 2000;287:408. However, humans do not have a corresponding Crry gene, the majority of women suffering from recurrent miscarriages do not have anti-phospholipid antibodies, and the underlying mechanism or mechanisms for recurrent miscarriages are still unclear. Further, since C3 is a key component of many pathways and mechanisms in vivo, including the classical, lectin and alternative pathways, and also has a role in clearing bacteria, other pathogens and immune complexes, specifically targeting C3-conversion in a pregnant woman could be associated with numerous unwanted and potentially risky side effects. Hence, knowledge of the role of individual components in the three pathways and identification of relevant pathways and of more precise targets for treatment could allow for new and improved treatment strategies.

Thus, there is a need for methods to prevent or reduce the risk of miscarriages, especially recurrent miscarriages, by safe and efficient methods. The invention addresses these and other needs in the art.

### SUMMARY OF THE INVENTION

The present invention is based on the discovery that complement activation is an effector in miscarriages, especially recurrent spontaneous miscarriage, and that inhibitors of specific components of the complement system may prevent or reduce the risk of miscarriage.

Accordingly, the present invention provides a method of preventing a miscarriage which comprises administering to a human female subject who is pregnant or planning to become pregnant an effective amount for preventing miscarriage in said female of an agent capable of inhibiting a component of the complement activation pathway. Preferably, the component is a member of the group consisting of factor B, factor D, properdin, C2, C3, C3 convertase, C4, C5, C5 convertase, C3a, C5a, membrane attack complex (MAC), C3a receptor, C5a receptor and members of the mannan-binding protein (MBL) pathway.

In one embodiment, the method comprises inhibiting C3 conversion with the agent. In this embodiment, the agent may comprise, for example, a member of the group consisting of an antibody directed against C3, and antibody directed against C3 convertase, and a cyclic peptide inhibitor having the amino acid sequence of SEQ ID NO:1. The antibody can be, e.g., a chimeric antibody, a humanized antibody, or a human antibody, as well as fragments thereof.

In another embodiment, the method comprises inhibiting C5 cleavage with the agent. In this embodiment, the agent may comprise, for example, a member of the group consisting of an antibody directed against C5 and an antibody directed against C5 convertase. The antibody can be, e.g., a chimeric antibody, a humanized antibody, or a human antibody, as well as fragments thereof.

In yet another embodiment, the method comprises inhibiting C5a receptor signaling with the agent. In this embodiment, the agent may comprise, for example, a member of the group consisting of an antibody directed against C5a, an antibody directed against the C5a receptor, and AcPhe(L-ornithine-Pro-D-cyclohexylalanine-Trp-Arg) (SEQ ID NO:2). The antibody may be, e.g., a chimeric antibody, a humanized antibody, or a human antibody, as well as fragments thereof.

In an additional embodiment, the method comprises inhibiting factor B, factor D or properdin capacity to activate the alternative pathway function. The agent may comprise, for example, an antibody directed against factor B or factor D. The antibody can be, e.g., a chimeric antibody, a humanized antibody, or a human antibody, as well as fragments thereof. A preferred antibody for factor B is MAb A1379, functional fragments(s) thereof, or antibodies that compete for factor B binding with MAb A1379.

In still another embodiment, the agent comprises an anti-sense nucleic acid sequence capable of binding to a nucleic acid encoding factor B, factor D, properdin, C2, C3, C3 convertase, C4, C5, C5 convertase, C3a, C5a, membrane attack complex (MAC) and the C3a or C5a receptors as well as certain members of the mannan-binding protein (MBL) pathway such as MBL-associated serine protease 1 or 2, or a small molecule capable of inhibiting the activity of the expression product of these nucleic acids.

The female subject may either be planning to become pregnant, or may already be pregnant and at risk for miscarriage. In one embodiment, the female subject has had at least one previous miscarriage, which was not caused by a genetic, anatomic, endocrine, or infectious condition.

The present invention also provides a method of preventing a miscarriage which comprises administering to a female subject who is pregnant or planning to become pregnant an effective amount for preventing miscarriage in said female of an agent capable of inhibiting a component of the alternative complement activation pathway. Preferably, the component is a member of the group consisting of factor B, factor D and properdin. The female subject may be human or another mammal, and may or may not suffer from APS.

The invention also provides for a method of screening to identify an agent useful for treating or preventing miscarriage which comprises (i) providing a pool of test agents; (ii) determining whether any test agent from the pool inhibits the activity of at least one member selected from the group consisting of factor B, factor D, properdin, C2, C3, C3 convertase, C4, C5, C5 convertase, C3a, C5a, membrane attack complex (MAC) and the C3a or C5a receptors as well as certain members of the mannan-binding protein (MBL) pathway such as MBL-associated serine protease 1 or 2, and (iii) selecting any test agent from the pool that inhibits the activity of at least one member as an agent useful for treating or preventing miscarriage. In one embodiment, the method comprises a step of selecting the pool of test agents prior to step (i). In another embodiment, the determining step comprises the steps of: (a) measuring the level of a complement split product formed downstream from the at least one member; (b) comparing the level of the complement split product to a control value; and (c) selecting any test agent for which the level of the complement split product is higher than the control value as an agent useful in treating or preventing miscarriage.

The above features and many other attendant advantages of the invention will become better understood by reference to the following detailed description when taken in conjunction with the accompanying drawings.

### BRIEF DESCRIPTION OF THE DRAWINGS

FIG. 1 shows fetal resorption rates for DBA/2-mated CBA/J mice (CBA/J×DBA/2), a murine model of spontaneous recurrent miscarriage, as compared to controls. The resorption rate was calculated as number of resorptions per total number of formed fetuses and resorptions. Statistically significant differences were observed between CBA/J×DBA/2 and CBA×Balb/c mice (p<0.01) and between CBA/J×DBA/2 and Balb/c×Balb/c mice (p<0.01).

FIG. 2 shows that blockade of complement effectors prevents fetal loss in CBA/J×DBA/2 mice. Pregnant mice (n=6-20 per group) were treated with an inhibitor of C3 convertase, Crry-Ig (3 mg i.p. on days 8, 10 and 12), or with monoclonal anti-C5 mAb (1 mg ip on days 8 and 10). Statistically significant differences were observed between CBA×DBA versus CBA×Balb/c (p<0.01); CBA×DBA+Crry; and CBA×DBA+anti-C5 MAb.

FIG. 3 shows that activating FcγRs are not required for aPL antibody-mediated pregnancy loss. Pregnant FcRγ^{+/+} and FcRγ^{-/-} mice were treated with IgG (10 mg i.p.) from a healthy non-autoimmune individual (NH-IgG), three different patients with APS (aPL-IgG), F(ab)′₂ fragments from a pool of aPL-IgG from patients 2 and 3 [aPL-F(ab)′₂], or human monoclonal aPL antibody (aPL mAb) on days 8 and 12 of pregnancy. Mice were sacrificed on day 15 of pregnancy, uteri were dissected, fetuses were weighed, and frequency of fetal resorption calculated (number of resorptions/number of fetuses+ number of resorptions). There were 4-7 mice in each experimental group. Treatment with all intact aPL-IgG preparations and aPL mAb caused an increase in fetal resorptions in FcRγ^{+/+} (*P<0.05 vs. NH-IgG, Student's t-test). Administration of aPL-F(ab)′₂ did not affect pregnancy outcome. FcRγ^{-/-} were not protected from fetal loss induced by intact aPL-IgG (*P<0.05 vs. NH-IgG, Student's t-test). In surviving fetuses from FcRγ^{-/-}, there was 36% decrease in weight.

FIGS. 4A-C show that C4 or C5 deficiency prevents aPL antibody-induced fetal loss and growth restriction. (A) Pregnant C4+/+ and C4−/− mice were treated with aPL-IgG (10 mg i.p.) or NH-IgG on days 8 and 12 of pregnancy and fetal resorption frequencies were determined on day 15 (n=5 mice/group). (*P<0.001, aPL vs control). (B and C) Pregnant C5+/+ and C5−/− mice were treated i.p. with aPL-IgG (10 mg), monoclonal human aPL antibody (1 mg), monoclonal human anti-DNA (1 mg) or their respective controls (NH-IgG, or monoclonal human anti-rabies antibody) on days 8 and 12 of pregnancy. Fetal resorption frequencies and fetal weights were determined on day 15 of pregnancy (n=5-11 mice/group). (B and C) C5−/− mice were protected from fetal loss (B) and growth restriction (C), whereas in the C5+/+ background strain aPL-IgG or aPL mAb caused pregnancy complications (*P<0.01, aPL vs control).

FIGS. 5A-B show that inhibition of C5 activation with anti-C5 mAb prevents aPL antibody-induced pregnancy complications. (A and B) Pregnant BALB/c mice were treated with aPL-IgG (10 mg i.p.) or NH-IgG (10 mg i.p.) at days 8 and 12 or pregnancy. Mice also received either anti-C5 mAb (1 mg i.p.) or control murine IgG (Ctrl-mAb) (1 mg i.p.) on days 8 and 10 (n=5-11 mice/group). Pregnancies were assessed as described in the legend for FIG. 1. Administration of anti-C5 mAb prevented fetal resorption (A) and growth restriction (B) (*P<0.05 vs. NH-IgG+Ctrl-mAb).

FIGS. 6A-D show that blockade of C5a-C5aR interaction protects pregnancies from aPL antibody-associated injury. (A and B) Pregnant BALB/c mice were given aPL-IgG (10 mg i.p.) or NH-IgG (10 mg i.p.) on days 8 and 12, and some also received C5aR antagonist peptide (C5aR-AP) (50 μg i.p.) on day 8, 30 minutes before administration of aPL-IgG (n=5-11 mice/group). Pregnancy outcomes were assessed as described in the legend for FIG. 3. Treatment with C5aR-AP prevented fetal loss and growth inhibition (*P<0.01, aPL vs. aPL+C5aR-AP). (C and D) The effects of effects of aPL-IgG on pregnancy outcomes in C5aR^{-/-} and C5aR^{+/+} mice were compared (n=5-11 mice/group). Pregnant mice were treated with aPL-IgG or NH-IgG as described above. C5aR^{-/-} mice were protected from aPL-IgC-induced fetal resorption and growth inhibition (*P<0.05, aPL-IgG vs. NH-IgG).

FIGS. 7A-B show that neutrophil depletion protects mice from aPL antibody-induced pregnancy complications and limits C3 deposition. BALB/c mice received anti-mouse granulocyte RB6-8C5 mAb (anti-Gr) (100 μg i.p.) or IgG2b isotype control mAb on day 7 of pregnancy. Depletion of neutrophils from the peripheral blood was observed 24 hours after administration of anti-Gr and persisted through day 15. On days 8 and 12, mice were treated with aPL-IgG (10 mg i.p.) or NH-IgG (10 mg i.p.) (n=5-11 mice/group). (A and B) Neutrophil depletion protected mice from fetal resorption (A) (*P<0.01, aPL-IgG+anti-Gr vs. aPL-IgG+IgG2b) and growth restriction (B) (*P<0.01, aPL-IgG+anti-Gr vs. aPL-IgG+IgG2b).

FIGS. 8A-B show that the absence of factor B protects mice from aPL antibody-induced fetal loss and extensive C3 deposition within deciduas. fB^{+/+} and fB^{-/-} mice were treated with aPL-IgG (10 mg i.p.) or NH-IgG (10 mg i.p.) on days 8 and 12 of pregnancy. Fetal resorption frequencies and fetal weights were determined on day 15 of pregnancy (n=4-8 mice/group). In contrast to fB^{+/+}, mice deficient in factor B were protected from fetal resorption (*P<0.05, fB^{+/+} aPL-IgG vs NH-IgG) and growth restriction (p<0.001, fB^{+/+} aPL-IgG vs NH-IgG ).

FIGS. 9A-B show that the mAb 1379 inhibits the alternative complement pathway in vitro. A zymosan assay using mouse serum (A) and an assay of rabbit erythrocyte lysis by human serum (B) were used to test the ability of mAb 1379 to inhibit alternative pathway activity. In the zymosan assay, 1 μg of the mAb resulted in significant inhibition and 1.5 μg resulted in nearly complete inhibition of the alternative pathway. In the rabbit erythrocyte lysis assay 1.5 μg of inhibitor resulted in significant inhibition, and 3 μg resulted in complete inhibition. *P<0.01 vs. 0 μg of inhibitor. **P<0.001 vs. 0 μg of inhibitor.

FIG. 10 shows the pharmacokinetics of inhibition of the alternative pathway by mAb 1379. Mice were given single intraperitoneal injection of one (-♦-) or two mg (-▪-) of the antibody. Sera was collected at various time points thereafter and tested for alternative pathway activity in the zymosan assay. A two mg injection led to complete inhibition of the alternative complement pathway for up to 48 hours.

FIGS. 11A-B shows that treatment with mAb 1379 prevents aPL Ab-mediated complement activation and pregnancy loss. Pregnant mice were given aPL-IgG or with normal human IgG on days 8 and 12 of pregnancy. Mice were also treated with either mAb 1379 or with control mouse IgG on days 7-13 of pregnancy. On day 15 of pregnancy the uteri were dissected. (A) Mice that received aPL Ab had a high rate of fetal resorption compared to those that received normal human IgG (P=0.001). However, treatment with mAb 1379 led to a significant reduction in the rate of fetal resorption compared to those mice receiving aPL and control IgG (20.3±6 vs. 40.5±8.7, P<0.01). (B) Immunohistochemical analysis was performed on decidual tissue from mice on day eight of pregnancy. aPL Ab caused extensive C3 deposition, but little C3 was seen in tissue from mice that were treated with the mAb 1379.

### DETAILED DESCRIPTION OF THE INVENTION

According to the present invention, anti-complement strategies or strategies down-regulating specific steps in the complement activation pathways can be employed to treat, reduce the risk of, or prevent miscarriages, especially recurrent miscarriages. For example, it has been found that complement activation not associated with APS or the presence of anti-PL antibodies can increase the risk of miscarriage. Thus, in one embodiment, the invention offers method to prevent further miscarriages for patients where genetic, anatomic, endocrine, and infectious causes of fetal damage have been ruled out as causes of the miscarriage, especially those patients which do not suffer from APS or have detectable levels of anti-PL antibodies in the circulation. In a second embodiment, the preferred patient group in which recurrent miscarriages can be prevented according to the invention are those suffering from “immunologically triggered miscarriages,” including those where no anti-PL antibodies has been found in the blood. In a third embodiment, an inhibitor of the alternative pathway is employed to treat or reduce the risk of miscarriages in a patient suffering from APS.

Preferably, these preventive strategies are based on the administration of one or more agonists or antagonists of specific components of the complement system, such as, but not limited to, factor B, factor D, properdin, C2, C3, C3 convertase, C4, C5, C5 convertase, C3a, C5a, membrane attack complex (MAC) and the C3a or C5a receptors as well as certain members of the mannan-binding protein (MBL) pathway such as MBL-associated serine protease 1 or 2. The common feature of all preventive/therapeutic strategies of the invention is that they reduce complement activation in a manner that can be measured by testing for the presence of certain complement “split-products,” i.e., peptides resulting from the cleavage or activation of complement components. Successful reduction or inhibition of the complement pathway would thus lead to a reduced amount or concentration of these split-products in blood. Complement split-products include C3a, C3d, C4a, Bb, Ba, C4d, and SC5b-9 and reduced concentrations of Bb and Ba, as well as C3d and C3a indicate factor B inhibition.

The experiments in murine models reported herein describe a critical role for complement regulatory proteins in feto-maternal tolerance and show that complement activation is required for pregnancy loss, especially antiphospholipid-induced pregnancy loss. For example, Example 1 shows that complement activation products are detectable in immune-mediated pregnancy loss, and that inhibition of C3 convertase activity using a C3 convertase inhibitor, inhibition of C5 cleavage using an anti-C5 antibody, and inhibition of C5a binding to its receptor using a peptide antagonist of C5aR, all prevented pregnancy loss. Example 2 confirms the results of Example 1, and also shows that lack of C4 or factor B prevents pregnancy loss. Example 3 shows that inhibition of the alternative pathway using an anti-factor B antibody was capable of preventing pregnancy loss.

According to the invention, targeting of complement components with inhibitory compounds can be used to prevent fetal loss, especially recurrent fetal loss. Notably, blocking the complement cascade at C5 may be especially advantageous since it would inhibit mediators and effectors of tissue injury while preserving the complement-derived immunoprotective functions of C3. Similarly, a factor B inhibitor would leave the classical pathway intact, so the use of this type of inhibitor poses less risk of serious infection than inhibitors that also block the classical pathway, would allow immune complex processing that is dependent on C4 and C3. Preferred patient groups include both females suffering from APS as well as females not suffering from APS. The present invention also provides for screening methods to identify additional agents that could be used in preventing miscarriage based on their capability to inhibit the activity of specific components of the classical, alternative and lectin pathways.

### Definitions

The following defined terms are used throughout the present specification, and should be helpful in understanding the scope and practice of the present invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value.

A “subject” or “patient” is a female human or an animal which is likely to have a miscarriage, or which have experienced at least one miscarriage. The subject may be pregnant or a pregnancy may be planned. The animal is a mammal, preferably a rodent or a primate.

The term “treatment” means to therapeutically intervene in a pregnant female or a female planning a pregnancy. The term “treatment” also encompasses prevention, which means to prophylactically interfere with a pathological mechanism that would or would likely result in miscarriage.

The “activity” of a member of the complement pathway means the ability of the member to promote complement activity downstream from the member. For example, the activity of C3 and C5 is the ability to being converted by C3 and C5 convertase, respectively; the activity of C3 and C5 convertase is the ability to convert C3 and C5, respectively; the activity of C5a is its ability to bind to the C5a receptor; the activity of the C5a receptor is its ability to promote activation of neutrophils, monocytes, and mast cells upon binding a C5a receptor ligand, and the activity of factor B, factor D and properdin is to activate C3 and then C5 as well as to generate complement activation fragments.

The term “modulating complement activity” in a subject means modifying it so that it is rendered as close as possible to the normal complement activity of a control subject. It especially encompasses inhibiting, i.e., reducing to a normal level, the complement system activity in patients at risk for, or suffering from, recurrent miscarriages.

As defined herein, a woman suffering from “recurrent miscarriages” has had more than one miscarriage, or a woman suffering from a disorder known to result in recurrent miscarriages.

The term “therapeutically effective amount” is used herein to mean an amount or dose sufficient to modulate, e.g., decrease the level of complement activity to the level of a normal subject. Preferably, a therapeutically effective amount can prevent or reduce the risk of a miscarriage. Alternatively, a therapeutically effective amount is sufficient to cause an improvement in a clinically significant condition in the host, such as increased complement activity.

The substance that modulates or inhibits complement activity is advantageously formulated in a pharmaceutical composition, with a pharmaceutically acceptable carrier. This substance may be then termed the active ingredient or therapeutic agent for prevention of miscarriages, particularly recurrent miscarriages.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered.

In the present invention there may be employed conventional molecular biology, microbiology, and recombinant DNA techniques within the skill of the art. The general genetic engineering tools and techniques discussed herein, including transformation and expression, the use of host cells, vectors, expression systems, etc., are well known in the art. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al. 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

### Inhibition of Complement Activation

The complement system of humans and other mammals involves more than 20 components and has a wide array of functions associated with a broad spectrum of host defense mechanisms including anti-microbial and anti-viral actions (Muller-Eberhard, Annu. Rev. Biochem. 1988;57:321-347; Rother et al., In: “Contemporary Topics in Immunology,” Vol. 14 (Snyderman, R., Ed.), pp. 109-153, Plenum Publishing Company, New York (1984)). The activation pathway for the complement system includes the classical pathway, the alternative pathway, and the mannan-binding lectin pathway which are initiated and regulated by separate mechanisms, but which all lead to the formation of components C5 to C9 and the resulting membrane attack complex (MAC), which lyses and destroys the cell under attack. Complement proteins are believed to contribute to certain types of tissue damage in, e.g., autoimmune diseases, and various soluble complement regulatory molecules, anti-complement component antibodies, anti-complement receptor antibodies, and peptide antagonists of complement receptors have been used or proposed as therapeutic agents in rheumatoid arthritis patients, post-acute myocardial infarction patients, patients who suffer strokes, cardiopulmonary bypasses, systemic lupus nephritis patients, and patients receiving new transplanted organs.

According to the invention, miscarriage, especially miscarriage triggered by other immunological mechanisms than APS, can be prevented by the administration to the pregnant mammalian female of modulators of the complement system. The common feature of these modulators or therapeutic agents is that they all lead to reduced complement activation, thereby reducing the risk for miscarriage. Such agents may be inhibitory antibodies or antibody-fragments, anti-sense nucleic acids or inhibitory peptides, or small organic or inorganic compounds that are capable of specifically inhibiting the activity of a target molecule.

In one embodiment, known inhibitors of complement activation are tested and/or applied for preventing pregnancy loss or recurrent pregnancy loss in animal models or human patients. For example, a 13-residue cyclic peptide called Compstatin that inhibits C3 cleavage by binding to C3 has been developed (Morikis et al., Protein Sci 1998;7:619-27; U.S. Pat. No. 6,319,897 to Lambris et al.). The peptide has the sequence of: Ile Cys Val Val Gln Asp Trp Gly His His Arg Cys Thr (SEQ ID NO:1).

In one embodiment, the therapeutic agents or modulators block or inhibit C5 activity (i.e., cleavage) or C5a-R signaling. The C5a-R is a hepta-helical seven-transmembrane spanning protein, which, upon binding by C5a or another C5a-R agonist, mediates the pro-inflammatory and chemotactic actions of, e.g., neutrophils, monocytes and mast cells (Gerard and Gerard, Nature 1991;349:614-617). For inhibition of C5, a humanized monoclonal antibody C5 Inhibitor 5G1.1 developed by Alexion Pharmaceuticals (Cheshire, CT) is currently in Phase II clinical trials to investigate its potential anti-inflammatory effect in, e.g., rheumatoid arthritis and membranous nephritis patients. The antibody stops the cleavage of C5 into fragments (C5a/C5b). In addition, several antagonists of C5a have been developed. For C5a as the drug target, the C5a-R antagonist AcPhe(L-ornithine-Pro-D-cyclohexylalanine-Trp-Arg) (SEQ ID NO:2) can be used, and additional potent C5a antagonists can be prepared from C5a peptides using, e.g., the techniques described in U.S. Pat. Nos. 5,942,599 and 5,696,320.

Complement factor D (Enzyme Commission No. 3.4.21.46, Enzyme Nomenclature, (1973) American Elsevier, New York)), an enzyme of the serine proteinase family, is believed to be the rate limiting enzyme for activation of the complement system via the alternative pathway. The structure and activation of this protein has been characterized, with a very low catalytic activity towards synthetic esters (Kim et al., Biochemistry 1994;33:14393-9; Jing et al., J Mol Biol 1998;282:1061-81). Accordingly, various synthetic esters can be designed and screened for optimal inhibition of Factor D activity.

Various other methods and compositions for inhibiting complement activation that are known and used in the art are described in Sahul and Lambris (Immunopharmacol 2000;49:133-148), and in, e.g., U.S. Pat. Nos., 5,135,916; 5,635,178; 5,650,389; 5,627,264 5,955,441; 5,989,592, 6,232,296; and 6,551,595, all of which are hereby incorporated by reference in their entireties. While inhibitors or agonists against any complement component described herein can be used, those inhibiting “down-stream” complement components or components specific to the activation or progression of the complement activation pathway are preferred, as they are likely to be associated with fewer unwanted side-effects.

In a further embodiment, the modulatory agent may be a candidate drug as identified by a screening method, an inhibitory antibody directed against any one of the components, or an antisense nucleic acid. All of these embodiments are described in greater detail below.

Other complement activation inhibitory agents, including antibodies and anti-sense polynucleotides, as well as methods to identify the agents and to apply them in a therapeutic regiment to reduce the risk of miscarriage, are described below.

### Inhibitory Antibodies

The modulatory substance may also be an antibody that is directed against a component of the complement system. Antibodies that block the activity of a component may be produced and selected according to any standard method well-known by one skilled in the art.

According to the invention, polypeptide fragments of selected components of the complement system, e.g., factor B, factor D, properdin, C2, C3, C3 convertase, C4, C5, C5 convertase, C3a, C5a, membrane attack complex (MAC) and the C3a or C5a receptors as well as certain members of the mannan-binding protein (MBL) pathway such as MBL-associated serine protease 1 or 2, may be produced, e.g., by recombinant techniques or by chemical synthesis, and fragments or other derivatives or analogs thereof, including fusion proteins, may be used as an immunogen to generate antibodies that recognize the polypeptide. Preferred polypeptides are those known to be involved in the activity of the complement component or its binding to other components or regulators. In particular embodiment, the antibody inhibits factor B interaction with factor D, leading to inhibition of factor B cleavage and the subsequent C3 activation. Factor B inhibition by an antibody or other antagonist can be measured, e.g., by a zymosan assay or an assay based on inhibition of lysis of unsensitized rabbit erythrocytes, both of which assays are described in Example 3. A preferred antibody for use in the methods described herein is MAb A1379, functional fragments thereof (including Fab′ and (Fab′)₂ fragments), and antibodies competing with binding of MAb A1379 binding to Factor B. The production and characterization of MAb A1379 is described in co-pending U.S. application 60/543,594, filed on Feb. 4, 2004, which is hereby incorporated by reference in its entirety.

The antibodies include but are not limited to, polyclonal, monoclonal, chimeric, single chain, Fab fragments, and a Fab expression library. Various procedures are known for the production of polyclonal antibodies. For example, various host animals can be immunized by injection with the polypeptide, or a derivative (e.g., fragment or fusion protein) thereof, including but not limited to rabbits, mice, rats, sheep, goats, etc. In one embodiment, the polypeptide is conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols and polyanions, oil emulsions, keyhole limpet hemocyanins, dinitrophenol, and potentially useful human adjuvants such as BCG (Bacillus Calmette-Guerin) and Corynebacterium parvum.

For preparation of monoclonal antibodies, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridoma technique (Kozbor et al., Immunology Today 1983, 4:72; Cote et al., Proc. Natl. Acad. Sci. U.S.A. 1983, 80:2026-2030), and the EBV-hybridoma technique to produce human monoclonal antibodies (Cole et al., in Monoclonal Antibodies and Cancer Therapy, Alan R. Liss, Inc., 1985, pp. 77-96). In an additional embodiment of the invention, monoclonal antibodies can be produced in germ-free animals (International Patent Publication No. WO 89/12690). According to the invention, techniques developed for the production of “chimeric antibodies” (Morrison et al., J. Bacteriol. 1984, 159:870; Neuberger et al., Nature 1984, 312:604-608; Takeda et al., Nature 1985, 314:452-454) may also be used. Briefly, such techniques comprise splicing the genes from an antibody molecule from a first species of organism (e.g., a mouse) that is specific for the polypeptide together with genes from an antibody molecule of appropriate biological activity derived from a second species of organism (e.g., from a human). Such chimeric antibodies are within the scope of this invention. In another embodiment, the construct is derived from a humanized antibody, in which the CDRs of the antibody (except for the one or more CDRs containing the heterologous binding sequence) are derived from an antibody of a non human animal and the framework regions and constant region are from a human antibody (see, U.S. Pat. No. 5,225,539; Oi et al., supra). The creation of completely human monoclonal antibodies is also possible through, e.g., the use of transgenic mice in which the mouse immunoglobulin gene loci have been replaced with human immunoglobulin loci to provide in vivo affinity-maturation machinery for the production of human immunoglobulins.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

According to the invention, techniques described for the production of single chain antibodies (U.S. Pat. Nos. 5,476,786, 5,132,405, and 4,946,778) can be adapted to produce polypeptide-specific single chain antibodies. An additional embodiment of the invention utilizes the techniques described for the construction of Fab expression libraries (Huse et al., Science 1989, 246:1275-1281) to allow rapid and easy identification of monoclonal Fab fragments with the desired specificity for a CK 2 polypeptide, or its derivatives, or analogs.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a polypeptide, one may assay generated hybridomas for a product which binds to a polypeptide fragment containing such epitope. For selection of an antibody specific to a polypeptide from a particular species of animal, one can select on the basis of positive binding with polypeptide expressed by or isolated from cells of that species of animal.

### Antisense Therapy

In another embodiment, vectors comprising a sequence encoding an antisense nucleic acid according to the invention may be administered by any known methods, such as the methods for gene therapy available in the art. Exemplary methods are described below. For general reviews of the methods of gene therapy, see, Goldspiel et al., Clinical Pharmacy 1993, 12:488 505; Wu and Wu, Biotherapy 1991, 3:87 95; Tolstoshev, Ann. Rev. Pharmacol. Toxicol. 1993, 32:573-596; Mulligan, Science 1993, 260:926-932; and Morgan and Anderson, Ann. Rev. Biochem. 1993, 62:191-217; May, TIBTECH 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY.

### RNA Interference (RNAi or siRNA)

In yet another embodiment, RNA interference technology can be used. The inhibitor of complement may take the form of an siRNA. It has recently been demonstrated that expression of selected genes can be suppressed in human cells by transfecting with exogenous, short RNA duplexes (siRNA) where one strand corresponds to a target region of the MRNA, i.e., EST of interest (Elbashir et al., Nature 2001; 411:494-498). The siRNA molecules are typically greater than 19 duplex nucleotides, and upon entry into the cell, siRNA causes the degradation of single-stranded (ssRNAs) RNAs of identical sequences, including endogenous mRNAs. siRNA is more potent than standard anti-sense technology since it acts through a catalytic mechanism. Effective strategies to deliver siRNAs to target cells in cell culture include physical or chemical transfection. An alternative strategy uses the endogenous expression of siRNAs by various Pol III promoter expression cassettes that allow transcription of functional siRNAs or their precursors (Scherr et al., Curr. Med. Chem. 2003;10(3):245-56). Recently, the RNA-polymerase III dependent promoter (H1-RNA promoter) was inserted in the lentiviral genome to drive the expression of a small hairpin RNA (shRNA) against enhanced green fluorescent protein (Abbas-Turki et al., Hum. Gene Ther. 2002;13(18):2197-201). siRNA can also be delivered in a viral vector derived, e.g., from a lentivirus (Tiscornia et al., Proc. Natl. Acad. Sci. U.S.A. 2003; 100:1844-8). For review articles, see Hannon, Nature 2002; 418:244-51 and Bernstein et al., RNA 2001; 7(11):1509-21. This technology also has been described in vitro in cultured mammalian neurons in Krickevsky and Kosik, Proc. Natl. Acad. Sci. USA 2002; 99(18):11926-9. siRNA technology is also being used to make transgenic animals (Cornell et al., Nat. Struct. Biol. 2003;10(2):91-2). RNA is described in Publication Nos. WO 99/49029 and WO 01/70949.

In one embodiment, a vector is used in which the coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA 1989, 86:8932 8935; Zijlstra et al., Nature 1989, 342:435 438).

Delivery of the vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy.

In a specific embodiment, the vector is directly administered in vivo, where it enters the cells of the organism and mediates expression of the construct. This can be accomplished by any of numerous methods known in the art and discussed above, e.g., by constructing the vector as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell surface receptors or transfecting agents, encapsulation in biopolymers (for examples, see, e.g., U.S. Pat. No. 5,635,493), encapsulation in liposomes, microparticles, or microcapsules; by administering it in linkage to a peptide or other ligand known to enter the nucleus; or by administering it in linkage to a ligand subject to receptor mediated endocytosis (see, e.g., Wu and Wu, J. Biol. Chem. 1987, 62:4429 4432), etc. In another embodiment, a nucleic acid ligand can be attached to a fusogenic viral peptide which is capable of disrupting endosomes or lysosomes, thereby avoiding lysosomal degradation of the nucleic acid. Cationic 12-amino acid peptides, e.g., derived from antennapedia, can also be used to facilitate the transfer of the nucleic acid into cells (Mi et al., Mol. Therapy 2000, 2:339 47). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188).

Preferred anti-complement component antisense sequences are those that are 8-30 nucleotides in length, and are complementary to at least one portion of factor B, factor D, properdin, C2, C3, C3 convertase, C4, C5, C5 convertase, C3a, C5a, membrane attack complex (MAC) and the C3a or C5a receptors as well as certain members of the mannan-binding protein (MBL) pathway such as MBL-associated serine protease 1 or 2 cDNA or MRNA. For sequence information, see, e.g., GenBank accession Nos. M65134, NM_{—}001736, and NM_{—}001710

### Screening Methods

Various methods for measuring products or components of the complement system, e.g., C3a, C4a, C5a, factor B and their derivatives, are known in the art and include, for example, radioimmunoassays (see, e.g., Hugli et al., In: “Immunoassays: Clinical Laboratory Techniques for the 1980s,” 443-460, Alan R. Liss, Inc., New York, N.Y. (1980), and Wagner et al. Analyt. Biochem. 1984;136:75-88). Methods for measuring and analyzing complement activation have also been described in the patent literature, describing, e.g., use of serine protease inhibitors (U.S. 6,297,024), antibodies (U.S. 4,624,482) and ELISA (4,722,890), and the addition of substrates for a particular complement convertase (U.S. 6,087,120). Commercial kits are also available from, for example, Amersham Biosciences (Piscataway, NJ) and Quidel (San Diego, CA). See also Larsson and Sjoquist, J Immunol Methods 1989;1 19:103-9 and U.S. Pat. No. 4,731,336. Further, various methods for detecting complement split products are known in the art. For example, the National Jewish Medical & Research Center has established sensitive techniques for complement split products based on, e.g., radioimmunoassay (RIA) and enzyme-linked immunosorbent assay (ELISA) techniques (see, World Wide Web (www) address at nationaljewish.org, “Complement Assays”, accessed May 5, 2003). The above-described methods and kits can be applied to screen for agents that modulate, preferably down-regulate, complement activation. The agents that are identified are potentially useful for treatment of miscarriages, including recurrent miscarriages.

A “test substance” or “test agent” is a chemically defined compound or mixture of compounds (as in the case of a natural extract or tissue culture supernatant), whose ability to modulate the binding or activity of a complement component may be defined by various assays. A “test substance” is also referred to as a “candidate drug” in the present description.

Test substances may be screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from, e.g., Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., TIBTech 1996, 14:60).

In one embodiment, the inhibitory molecule is a small molecule which can readily distribute in tissues after intravenous administration. For example, after generating and characterizing inhibitory antibodies, molecular modeling and rational molecular design can be used to generate small molecules which mimic the molecular structures of the binding region of the antibodies (i.e., the variable region). These small molecules can be peptides, peptidomimetics, oligonucleotides, or organic compounds. The mimicking molecules can be used as inhibitors of complement activation.

Identification of candidate substances can be achieved using any suitable assay, including without limitation (i) assays that measure selective binding of test compounds to the complement component (ii) assays that measure the ability of a test substance to modify (i.e., inhibit) a measurable activity or function of the complement component, and (iii) assays that measure the ability of a substance to modify (i.e., inhibit) the transcriptional activity of sequences derived from the promoter (i.e., regulatory) regions of the complement component gene.

For screening assays intended to identify agents which modulate or inhibit the expression of one or more components of the complement system, an in vitro method using a recombinant reporter gene promoter activity system can be used. In such a method, the expression of the complement gene of interest is linked to the expression of a reporter gene, which expression can be easily detected and/or quantified. Reporter genes for use in the invention encode detectable proteins, and include, but are by no means limited to, chloramphenicol transferase (CAT), β-galactosidase (β-gal), luciferase, green fluorescent protein (GFP) and derivatives thereof, yellow fluorescent protein and derivatives thereof, alkaline phosphatase, other enzymes that can be adapted to produce a detectable product, and other gene products that can be detected, e.g., immunologically (by immunoassay).

Potential drugs directly interacting with the expressed protein may be identified by screening in high-throughput assays, including without limitation cell-based or cell-free assays. It will be appreciated by those skilled in the art that different types of assays, both in vitro, in vivo, high-throughput, or simple assays, can be used to detect different types of agents. For example, several methods of automated assays have been developed in recent years so as to permit screening of tens of thousands of compounds in a short period of time (see, e.g., U.S. Pat. Nos. 5,585,277, 5,679,582, and 6,020,141).

Intact cells or whole animals, e.g., expressing a gene encoding a selected component of the complement system, or which is a model of recurrent miscarriages can be used in screening methods to identify candidate drugs. In one series of embodiments, a permanent cell line is established. Alternatively, cells are transiently programmed to express a gene by introduction of appropriate DNA or mRNA.

A preferred animal model is the one used in the Examples to identify agents preventing miscarriages, namely DBA/2-mated CBA/J mice. In this model, normally having a rate of fetal loss in the order of 30%, agents may be tested for their efficacy in reducing the miscarriage rate, or in bringing the miscarriage rate down to the level of control animals, e.g., CBA×Balb/c or Balb/c×Balb/c. This and other animal models can also be used to evaluate and optimize treatment regimens, dosages, etc., for preclinical testing of each selected drug. Also, selected complement inhibitors may be studied for their effect on the levels of various complement components, preferably down-stream components, such as complement split products, in humans or other animals.

Selected agents may then be modified to enhance efficacy, stability, pharmaceutical compatibility, and the like. Structural identification of an agent may be used to identify, generate, or screen additional agents. For example, where peptide agents are identified, they may be modified in a variety of ways, e.g., to enhance their proteolytic stability.

### Therapeutic Applications

The present invention provides a method for treating, preventing, or reducing the risk of, miscarriages, especially recurrent miscarriages. The method comprises administering to a patient in need of such treatment an effective amount of a therapeutic agent that modulates complement system activity or signaling, together with a pharmaceutically acceptable carrier. For example, the modulator or therapeutic agent may be a C3-convertase inhibitor, an antibody against C5, an antibody against factor B or factor D, or an antagonist of the C5a receptor.

The concentration or amount of the active ingredient depends on the desired dosage and administration regimen, which can be developed by standard preclinical testing methods. Suitable dose ranges may include from about 0.01 mg/kg to about 100 mg/kg of body weight in one day. Alternatively, the dose administered is one that is capable of achieving at least 90%, preferably 95%, and more preferably 99% inhibition of the activity of the target molecule.

For example, when administering an antibody or antibody fragment directed against C5, the drug could be administered at least once, or at least once a week before pregnancy, and/or at least once, or once a week once conception has occurred, for as long as it is needed. In one embodiment, about 8-10 mg/kg antibody is administered weekly for 3-4 weeks, and thereafter bi-weekly, i.e., once every two weeks. Each dose of antibody or antibody fragment would preferably include about 1 pg to 50 mg, more preferably about 1 mg to 15 mg, and most preferably about 5-10 mg, of antibody per kg bodyweight. A similar therapy regimen could be employed for a C3 inhibitor such as Compstatin (see, supra).

In a therapy regimen employing a C5a receptor antagonist such as AcPhe(L-ornithine-Pro-D-cyclohexylalanine-Trp-Arg), the agent could be administered at least once, preferably at least once a day before pregnancy, more preferably once a week, and/or at least once a week once conception has occurred, for as long as it is needed. Each dose of agent would preferably include about 1 pg to 50 mg, more preferably about 0.5 mg to 50 mg, and most preferably about 5 to 10 mg, of peptide per kg bodyweight per kg bodyweight.

When employing an antibody or other antagonist against factor B, the agent could be administered at least twice per week. Each dose of antibody would preferably comprise about 1 pg to 50 mg, more preferably about 1 mg to 15 mg, and most preferably about 5-10 mg, of antibody per kg bodyweight.

Preferably, the therapeutically effective agent according to the invention is administered together with a pharmaceutically acceptable carrier. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A composition comprising a complement activation inhibitor according to the invention is preferably substantially free of contaminants. For example, a composition is substantially free of contaminating compounds when at least about 75%, more preferably 90%, and even more preferably 99%, by weight of the total composition is the active compound according to the invention. It is also preferred that a composition, which is substantially free of contamination, contain only a single molecular weight species having the activity or characteristic of the species of interest.

The pharmaceutical compositions may be added to a retained physiological fluid such as blood or synovial fluid. In another embodiment, the active ingredient can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

In yet another embodiment, the therapeutic compound can be delivered by a controlled release system. For example, a polypeptide may be administered using intravenous infusion with a continuous pump, in a polymer matrix such as poly-lactic/glutamic acid (PLGA), a pellet containing a mixture of cholesterol and the active ingredient (SilasticRTM; Dow Corning, Midland, Mich.; see U.S. Pat. No. 5,554,601) implanted subcutaneously, an implantable osmotic pump, a transdermal patch, liposomes, or other modes of administration.

According to the invention, the pharmaceutical composition of the invention can be introduced parenterally, transmucosally, e.g., orally (per os), nasally, or rectally, or transdermally. Parental routes include intravenous, intra-arteriole, intramuscular, intradermal, subcutaneous, intraperitoneal, intraventricular, and intracranial administration. Preferably, the administration is intravenous for large proteins such as antibodies or antibody fragment, whereas intravenous or oral administration is preferred for peptide or other low-molecular weight drugs.

### EXAMPLES

The following Example(s) are understood to be exemplary only, and do not limit the scope of the invention or the appended claims.

### Example 1

### Complement Activation in a Mouse Model

CBA/J×DBA/2 pregnancy has been extensively studied as a model of immune-mediated pregnancy loss and shares many features with human recurrent spontaneous miscarriage, particularly peri-implantation loss. Here, it is shown that complement is activated and C3 is deposited within the decidua in this murine model of miscarriage.

### Fetal Resorption Rates

Female mice were allowed to mate with previously isolated male mice. The groups in the present experiment comprised 7-20 animals, one group of DBA/2-mated CBA/J mice and two controls (CBA×Balb/c and Balb/c×Balb/c). After mating, the females were checked daily until the presence of a vaginal plug was confirmed (this time point was then defined as day 0.5 of pregnancy). The mice were sacrificed 15 days post-conception. Uteri were dissected and the frequency of fetal resorption was determined as the number of resorptions divided by total number of formed fetuses and resorptions. Resorption sites, resulting from the loss of a previously viable fetus, are easily identifiable by visual inspection.

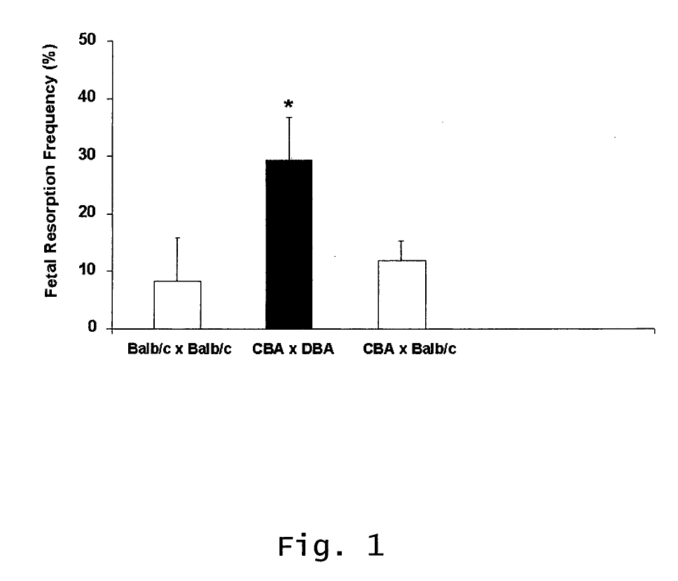

As shown in FIG. 1, the DBA/2-mated CBA/J mice displayed a 30% frequency of fetal resorption, 3-fold higher than that of control pregnancies.

### Complement Activation Products in Decidual Tissues

If complement is a mediator of fetal loss, complement activation products may be detectable in decidual tissues from DBA/2-mated CBA/J mice.

Mice were mated as described above, and sacrificed on day 8.5 post-conception. Sections of decidual tissue from CBA/J×Balb/c were incubated with an antibody to mouse C3, and followed by rabbit anti-goat IgG conjugated with HRP (horseradish peroxidase) and staining.

At day 8 post-conception, there was extensive C3 deposition throughout the decidua and extra-embryonic membranes, as well as necrosis, inflammatory infiltrates, and loss of embryonic elements (i.e., fetal debris). By contrast, there was no evidence of complement activation in decidua from control mice (CBA/J×Balb/c), as there was no staining for C3 as well as a normally developed embryo. These results show that complement activation is a mediator of fetal loss.

### Preventing Fetal Loss

This experiment was carried out to investigate if blockade of complement activation would prevent fetal loss in the murine model of miscarriage. In these experiments, DBA/2-mated CBA/J pregnant mice were treated with three different targeted inhibitors of complement activation.

Crry-Ig. Complement receptor 1-related gene/protein y (Crry) inhibits C3 convertase activity. The Crry used in the experiments was Crry-Ig, which is a recombinant soluble form of Crry fused to the hinge, CH2, and CH3 domains of mouse IgG1, prepared as described in Quigg et al., J Immunol 1998;160:4553-4560. Crry-Ig blocks activation of C3 by both the classical and alternative pathways, thereby preventing all complement effector activities. Pregnant DBA/2-mated CBA/J mice were treated with 3 mg Crry-Ig, administered i.p. on days 8, 10 and 12.

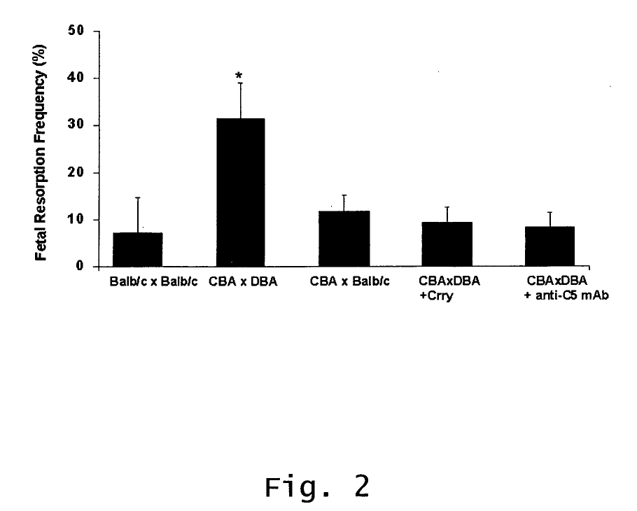

As shown in FIG. 2, DBA/2-mated CBA/J mice treated with the recombinant Crry-Ig protein showed nearly complete inhibition of complement activation in vivo, and treatment with Crry-Ig completely rescued pregnancies.

Monoclonal anti-C5 mAb. Also tested was an antibody against C5, which prevents the cleavage of C5. Pregnant DBA/2-mated CBA/J mice were administered 1 mg of the antibody i.p. on days 8 and 10.

As shown in FIG. 2, administration of the anti-C5 mAb prevented pregnancy loss in DBA/2-mated CBA/J mice. The anti-C5 antibody was equally effective as Crry-Ig in reducing pregnancy loss to control levels.

Peptide antagonist of C5aR. A peptide antagonist of C5aR, AcPhe(L-ornithine-Pro-D-cyclohexylalanine-Trp-Arg) (C5aR-AP), has been shown to prevents interaction between C5a and C5aR, thereby inhibiting activation of neutrophils, monocytes, and mast cells. Pregnant DBA/2-mated CBA/J mice were treated with 100 μg C5aR-AP, administered i.p. on day 8 after conception. Mice were sacrificed at day 15 of pregnancy.

Blockade of C5a-C5aR interaction prevented fetal loss in the CBA/J×DBA/2 model of miscarriage. A uterus from DBA/2-mated CBA/J mouse treated with C5aR-AP showed 6 larger fetuses with decidua and no resorptions, while a uterus from Balb/c-mated CBA/J mouse showed 7 fetuses with decidua, and a uterus from DBA/2-mated CBA/J mouse showed 2 small fetuses and 5 resorptions. The fetal resorption rate for mice treated with C5a-R antagonist was about 7%, showing successful prevention of miscarriage.

Blockade of factor B with mAb1379 prevented fetal loss in the CBA/J×DBA/2 model of miscarriage. To inhibit factor B, mice were injected on days 7 through 13 of pregnancy with mAb1379 (2 mg intraperitoneally) or with murine IgG as a control. Fetal resorption frequency was calculated on day 15 of pregnancy.

These experiments evidence that complement is critical in the pathogenesis of fetal loss, and that fetuses from DBA/2-mated CBA/J pregnant mice can be rescued by use of complement inhibitory therapies, in particular therapies directed against C3 or C5.

### Example 2

### Methods

This Example shows that factor B, C3, C5 and C5aR are required for pregnancy complications triggered by aPL antibodies, and that neutrophils are critical effectors cells in the model of APS. That aPL-IgG can initiate fetal damage in the absence of activating FcγRs, but not in the absence of C4, and that F(ab)′₂ fragments of aPL-IgG do not mediate such injury shows that initiation of the complement cascade can occur via the classical pathway. The observation that factor B is required for fetal death and that its presence is associated with increased C3 deposition shows that the alternative pathway amplifies local complement activation and also plays a critical role in the induction of fetal loss.

Mice. Adult mice (2-3 month old) were used in all experiments. BALB/c mice were purchased from Taconic Farms (Germantown, New York). FcRγ^{-/-} mice backcrossed to BALB/c mice were provided by Dr. Jeffrey Ravetch (Rockefeller University, New York, New York) (Takai et al., Cell 1994;76:519-529). C4^{-/-} mice were generated by homologous recombination and backcrossed to C57BL/6 for 17 generations (Wessels et al., Proc. Natl. Acad. Sci. USA 1995;92:11490-11494; Fischer et al., J. Immunol. 1996;157:549-556). C5^{-/-} (B10.D2-H2^{d}H2-T18^{c} Hc^{o}/o2Sn) and the C5^{+/+} background strain mice (B10.D2-H2^{d}H2-T18^{c} Hc^{o}/oSnJ) were obtained from Jackson Laboratories (Bar Harbor, Me.). C5aR deficientmice were generated by targeted deletion of the murine C5aR gene and determined to be completely C5a receptor deficient by PCR, Northern blot, and immunohistochemistry analyses. C5aR deficient animals were backcrossed with C57BL/6J mice. Heterozygous C5aR^{+/-} backcrossed mice were interbred and the resulting C5aR^{+/+} and C5aR^{-/-} littermates used for studies. Mice deficient in factor B were generated by targeted deletion (Matsumoto et al., Proc. Natl. Acad. Sci. USA 1997;94:8720-8724). FB^{-/-} mice were generated by intercrossing of fB^{+/-} and then maintained as a homozygous deficient strain. Procedures that involved mice were approved by the local Committee on Animal Use in Research and Education and were conducted in strict accordance with guidelines for the care and use if laboratory research animals promulgated by the NIH.

Preparation of aPL and other antibodies. Human IgG-containing aPL antibodies (aPL-IgG) were obtained from 3 patients with APS [characterized by high titer aPL antibodies (>140 GPL units), thromboses, and/or pregnancy losses] (1). IgG was purified by affinity chromatography using Protein G sepharose chromatography columns (Amersham Pharmacia Biotech, Piscataway, N.J.). Human IgG from healthy non-autoimmune individuals (NH-IgG) was purified by an identical method. All IgG samples were treated to deplete endotoxin with Centriprep ultracentifugation devices (Millipore Corporation, Bedford, Mass.) and determined to be free of endotoxin using the limulus amebocyte lysate assay. F(ab)′₂ fragments were obtained by digestion of purified aPL-IgG pooled from patients 2 and 3 using immobilized Pepsin (Pierce, Rockford, Ill.). The digested supernatants were passed through Protein G sepharose to remove remaining intact IgG, and their purity was assessed by Western blot using an antibody specific for the F(ab)′₂ fragment (Jackson ImmunoResearch, West Grove, Pa.). The F(ab)′₂ fragments were demonstrated to have similar antiphospholipid reactivity to the intact aPL-IgG by ELISA (Sigma Chemical, St. Louis, Mo.). The generation, structure and specificity of the human IgGI mAbs aPL (mAb 519), anti-DNA (mAB 412.67), and anti-rabies virus (mAb 57), were previously described (Ikematsu et al., Arthritis Rheum. 1998;41:1026-1039; Li et al., Eur. J. Immunol. 2000;30:2015-2026; Ikematsu et al., J. Immunol. 1993;150:1325-1337; Ikematsu et al., J. Immunol. 1998;161:2895-2905).

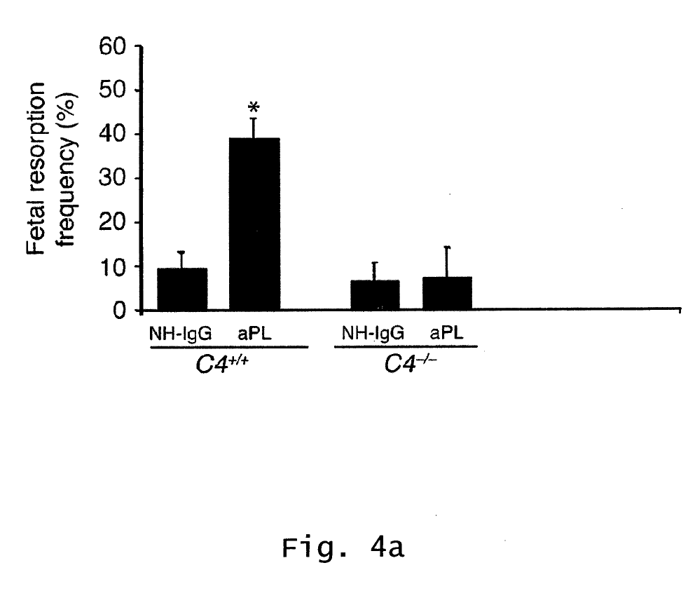

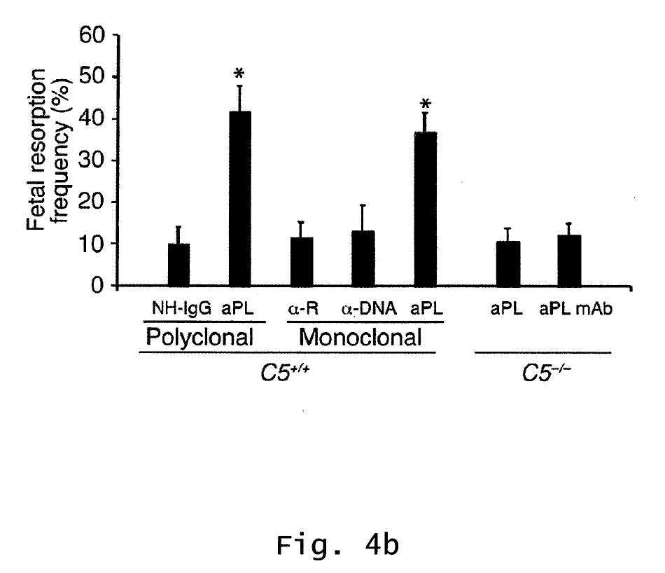

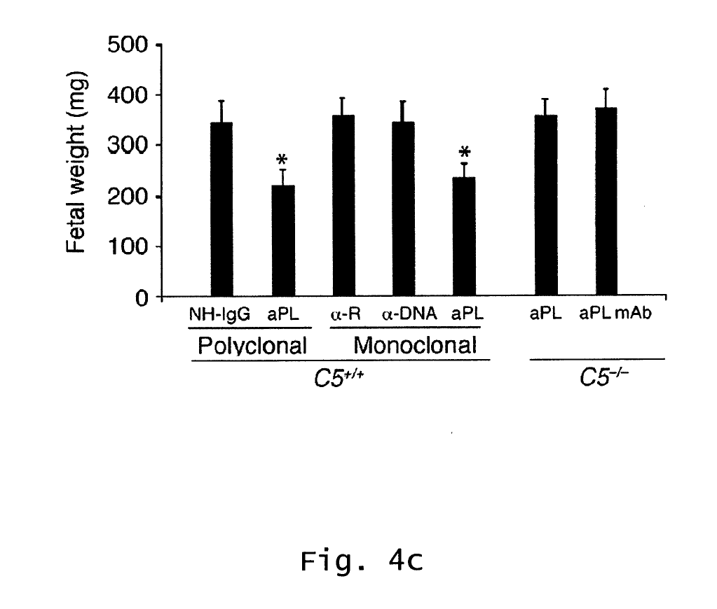

Murine pregnancy model. Females were mated with previously isolated males. Presence of a vaginal plug defined day 0 of pregnancy. On days 8 and 12 of pregnancy mice were treated with intraperitoneal (i.p.) injections of aPL-IgG (10 mg), aPL-IgG F(ab)′₂ (10 mg), NH-IgG (10 mg), human mAbs (aPL, anti-DNA, or anti-rabies) (1 mg) (Branch et al., Am. J. Obstet. Gynecol. 1990;163:210-216; Holers et al., J. Exp. Med. 2002;195:211-220). To inhibit C5, mice were treated on days 8 and 10 of pregnancy with anti-C5 mAb (1 mg i.p.) or murine IgG as a control (25). To block C5aR, mice received a C5aR antagonist peptide (AcPhe[L-ornithine-Pro-D-cyclohexylalanine-Trp-Arg]) (50 μg) on day 8, 30 minutes before treatment with aPL-IgG (Finch et al., J. Med. Chem. 1999;42:1965-1974; Mastellos et al., J. Immunol. 2001;166:2479-2486). To deplete neutrophils, mice were treated on day 7 with rat anti-mouse granulocyte RB6-8C5 mAb (Pharmingen, San Diego, Calif.) (100 μg, i.p.) that reacts with Ly6G (Gr-1 myeloid differentiation antigen); an IgG2b mAb was the isotype control. The level of Ly6G antigen expression in bone marrow correlates with granulocyte maturation; and in peripheral blood, rat anti-mouse granulocyte RB6-8C5 mAb recognizes neutrophils and eosinophils (Hestdal et al., J. Immunol. 1991;147:22-28; Lagasse et al., J. Immunol. Methods 1996; Conlan et al., J. Exp. Med. 1994;179:259-268). Neutrophil depletion was observed 24 hours after administration of anti-granulocyte mab and persisted through day 15. Mice were sacrificed on day 15 of pregnancy, uteri dissected, fetuses and placentas weighed, and fetal resorption rates calculated (number of resorptions/total number of formed fetuses and resorptions). Resorption sites are easily identified (see FIG. 4c) and result from loss of a previously viable fetus. Functional C3 activity in serum was measured using the previously described zymosan assay (Foley et al., Eur. J. Immunol. 1993;23:1381-1384).

Immunohistochemistry. Deciduas were removed from mice on day 8 of pregnancy, 60 minutes after administration of aPL-IgG, frozen in O.C.T. compound and cut into 10 μm sections. After quenching endogenous peroxidase with 1% H₂O₂ in methanol and blocking nonspecific binding sites with normal goat serum (Cappel, Aurora, Ohio), sections were incubated with goat anti-mouse C3, goat anti-human IgG, (Cappel), or goat anti-human F(ab′)₂ (Jackson ImmunoResearch) followed by anti-goat IgG conjugated to HRP (Sigma Chemical). To detect infiltrating granulocytes, sections were incubated with rat anti-mouse granulocyte RB6-8C5 mAb (Pharmingen) followed by rabbit anti-rat IgG conjugated to HRP (Sigma Chemical). Bound horseradish peroxidase (HRP) was detected with diaminobenzidine. Sections were counterstained with hematoxylin. The intensity of staining of C3 within decidual and embryo tissue was scored on a semiquantitative scale (0 to 5+) by three observers who were blinded to the experimental condition. Data are expressed as the mean of 5 -8 mice for each experimental condition. Sections of frozen tissue were also stained with H&E.

Western BlotAnalysis. Deciduas, including embryos, were removed from mice on day 8 of pregnancy, 60 minutes after administration of aPL-IgG, and immediately frozen at −70° C. The tissue was homogenized in RIPA lysis buffer containing 1% Triton X-100, 0.5% deoxycholic acid, 150 mM NaCl, 20 mM β-glycerophosphate, 20 mM Tris.HCl (pH 8.0), 5 mM EGTA, 3 mM MgCl₂, 0.1% SDS, 1mM DTT, 50 μM Na₃VO₄, and EDTA-free protease inhibitor cocktail (Roche Applied Science, Penzberg Germany). Lysates (50 μg/sample) were resolved by electrophoresis with a 10% Bis-Tris polyacrylamide gel (Invitrogen, Carlsbad, Calif.) and transferred to a nitrocellulose membrane. The membrane was probed with HRP-conjugated goat anti-mouse C3 (Cappel) and was visualized using a chemiluminescence detection kit (Amersham Life Science, Buckinghamshire, UK). The proteolytic cleavage product C3-alpha′ band was identified by comparing serum incubated with or without zymosan.

Statistical Analyses. Data are expressed as mean ± standard deviation. Student's t-test was used to compare fetal resorption rates and fetal weights between groups. Mann-Whitney U test was used to compare values for semiquantitative scoring of immunohistochemisty. A probability of less than 0.05 was used to reject the null hypothesis.

### Results

### Activating FcγR are not Required for aPL Antibody-induced Pregnancy Complications

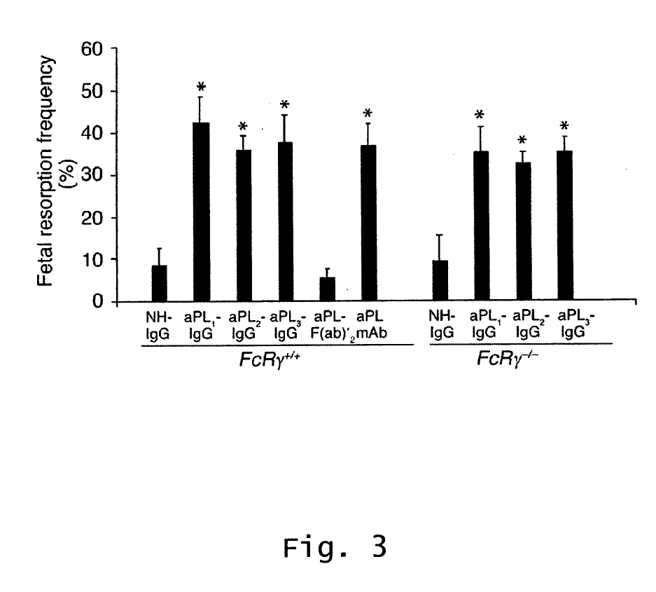

The Fc domain of pathogenic IgG can initiate tissue damage by binding FcγR on effector cells and/or initiating activation of complement. As a first approach to determine the role of FcγR in pregnancy loss induced by aPL antibodies, we compared the consequences of treating pregnant mice with polyclonal IgG isolated from APS patients and F(ab)′₂ fragments prepared from the same IgG source. Passive transfer of IgG from 3 different patients with high titer aPL antibodies (>140 GPL units) (aPL-IgG) consistently caused a 4-fold increase in the frequency of fetal resorption (FIG. 1a). In contrast, treatment with F(ab)′₂ fragments of aPL-containing IgG did not affect the frequency of fetal loss (FIG. 3a). Fetal loss in mice treated with F(ab)′₂ fragments of aPL-IgG was similar to that observed in mice treated with IgG from healthy individuals (NH-IgG) (FIG. 3a). In addition, growth restriction induced by treating pregnant mice with intact aPL-IgG was also absent in surviving fetuses of mice treated with aPL-IgG F(ab)′₂ [average fetal weight: aPL-IgG 213±42 mg, aPL-IgG F(ab)′₂343±43 mg, NH-IgG 326±32 mg; aPL-IgG F(ab)′₂ vs aPL-IgG, p<0.05]. Importantly, deposition of human F(ab)′₂ IgG in decidual tissues was similar in mice treated with aPL-IgG and aPL-IgG F(ab)′₂, and no human IgG deposition was observed in deciduas from mice treated with NH-IgG.

Given our finding that the Fc portion of IgG is necessary for aPL antibody-mediated injury, we considered the possibility that aPL antibodies deposited in the decidua initiate inflammation, thrombosis and fetal demise by crosslinking stimulatory FcγR expressed on monocytes, neutrophils, platelets or mast cells. To examine the role of FcγR in aPL antibody-induced pregnancy loss, we studied mice with targeted deletion of the common γ subunit (FcRγ^{-/-}) that is required for signaling by activating FcγR, high-affinity FcγRI and low affinity FcγRIII (Takai et al., Cell 1994;76:519-529). Although FcR γ-deficient mice are reported to have less severe or undetectable antibody-dependent experimental hemolytic anemia, thrombocytopenia, and glomerulonephritis (Ravetch et al., Annu. Rev. Immunol. 1998;16:421-432), we found that FcRγ^{-/-} mice were not protected from poor pregnancy outcomes after passive transfer of aPL-IgG (FIG. 1a). To exclude the possibility that FcRγ-deficiency altered the localization of aPL-IgG, we performed immunohistochemical analyses of deciduas from FcRγ^{+/+} and FcRγ^{-/-} at day 8 of pregnancy (harvested 60 minutes after treatment with aPL-IgG). Comparable amounts of human IgG were present in FcRγ-sufficient and FcRγ-deficient mice (FIGS. 3). Thus, in our murine model of APS, aPL-IgG targeted to the placenta can initiate fetal damage in the absence of activating FcγRs, while F(ab)′₂ fragments of aPL-IgG do not mediate such injury.

### Blockade of C4 or C5 Activation Protects Mice from aPL Antibody-induced Pregnancy Loss

The complement pathway presents a second Fc-dependent means of effecting antibody-mediated injury, and our initial studies showed that blocking C3 prevents fetal loss in murine APS (Holers et al., J. Exp. Med. 2002;195:211-220). To assess the importance of the classical pathway of complement activation, we treated pregnant C4-deficient mice with aPL-IgG. C4^{-/-} mice were protected from fetal loss (FIG. 4a) and growth restriction (average fetal weight in aPL-IgG-treated mice: C4^{+/+} vs C4^{-/-}248±19 mg vs.413±30 mg, p<0.001, N=5 mice/group) suggesting that aPL antibodies trigger the complement cascade through either the classical or lectin pathways. That the classical pathway is required as an initiator of complement activation by aPL-IgG is supported by our finding that F(ab)′₂ fragments of aPL-IgG, which lack the Fe portion necessary to activate the classical pathway, do not cause pregnancy loss (FIG. 3).

Following initiation of the complement cascade, any of several complement activation fragment-derived ligand-receptor interactions could mediate fetal injury such as we have observed. To define which elements of the complement cascade mediate pregnancy loss, we initially focused on complement component 5 (C5). C5 is a pivotal member of the complement system as all three initiating pathways converge to activate C5 and two effector pathways lead from it. To determine whether activation of C5 is required for aPL-induced fetal loss, we treated pregnant C5-deficient and C5-sufficient mice with aPL-IgG, control IgG, human aPL monoclonal antibody (mAb), human anti-DNA mAb, or control human IgGI mAb. In C5^{+/+} mice, both APS patient-derived polyclonal aPL-IgG and human aPL mAb caused a 4-fold increase in the frequency of fetal resorption and a significant decrease in embryo weight as compared to control IgG (FIGS. 4b-c). Treatment with anti-DNA antibody, an autoantibody often present in patients with APS, had no effect on pregnancy outcome. That the results from experiments with human aPL mAb were similar to results obtained with polyclonal aPL antibodies also indicates that antibodies reactive with aPL, rather than xenoreactive antibodies, which may be present in polyclonal human IgG, are sufficient to initiate complement activation and fetal damage in this model.

In contrast to C5^{+/+} mice, mice lacking C5 were protected from aPL antibody-induced pregnancy complications (FIGS. 4b-c). Immunohistochemical analyses of deciduas from day 8 of pregnancy, obtained 60 minutes after treatment with aPL-IgG, showed extensive deposition of human IgG and C3 and focal necrosis and neutrophil infiltration in C5^{+/+} mice. In C5^{-/-} mice, there were no inflammatory infiltrates, and deciduas and embryos had normal morphology despite the presence of human IgG within decidual tissue. Importantly, in C5^{-/-} mice there was less C3 deposition, as shown by comparing representative sections and by grading the intensity of C3 staining in embryos and decidua from aPL-IgG treated mice on a semiquantitative scale (0-5+) (C5^{-/-} vs C5^{+/+}: 2.5±0.5 vs 4.3±0.6, p<0.001, n=5 mice/group). Taken together, these results demonstrate that C5 activation is a critical proximal effector for the induction of fetal loss by aPL antibodies, and implicate C5 activation and its downstream effects in amplifying local C3 deposition.

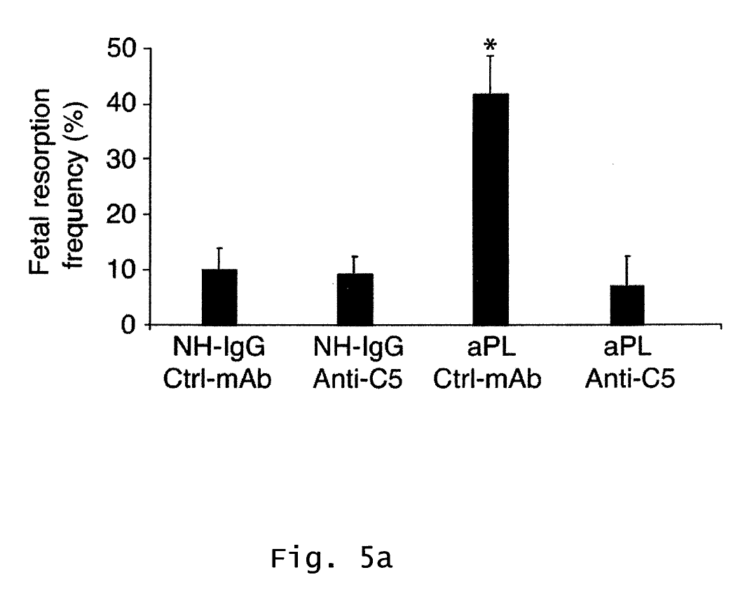

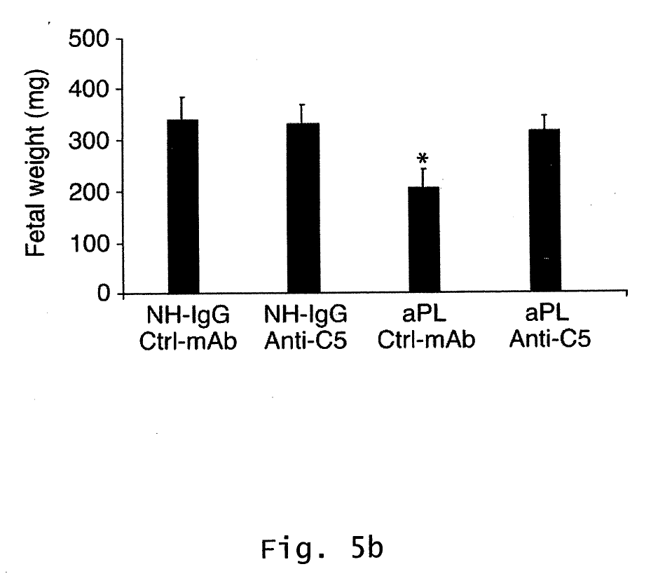

As an alternative strategy to confirm that C5 activation is required for fetal loss, we investigated the outcome of blocking C5 activation with anti-C5 mAb (Frei et al., Mol. Cell. Probes 1987;1:141-149). These experiments can prove particularly relevant because a similar anti-human C5 mAb is in phase II studies in patients with rheumatoid arthritis and phase I studies in active lupus nephritis (Quigg, R. J., Trends Mol. Med. 2002;8:430-436; Tesser et al., Arthritis Rheum. 2001;44: S274 (Abstr.)). We administered anti-C5 mAb prior to treatment with either NH-IgG or aPL-IgG. The ensuing blockade of C5 cleavage (Wang et al., Proc. Natl. Acad. Sci. 1995;92:8955-8959) prevented aPL antibody-induced pregnancy loss and growth restriction (FIGS. 5a and b). Indeed, pregnant mice treated with anti-C5 mAb were protected to a similar extent as C5-deficient mice (FIGS. 4b and c, FIG. 5).

### C5a-C5aR Interactions are Critical Mediators of aPL Antibody-induced Pregnancy complications

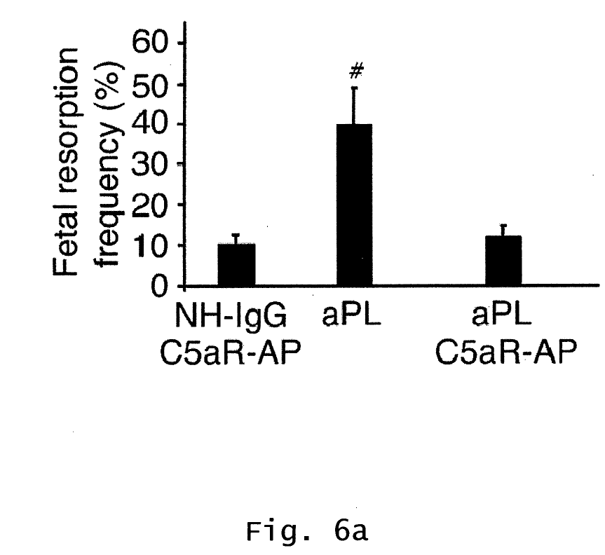

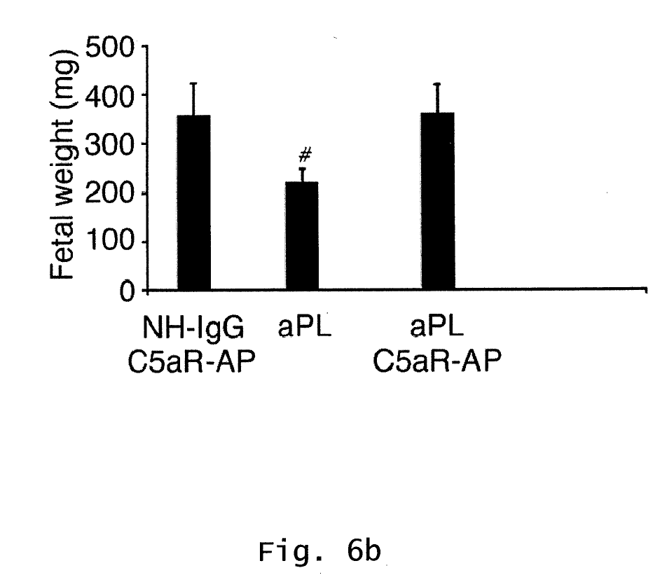

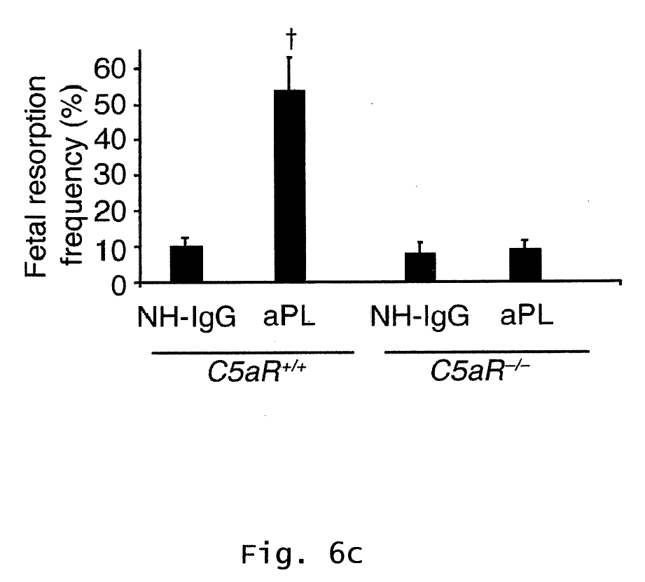

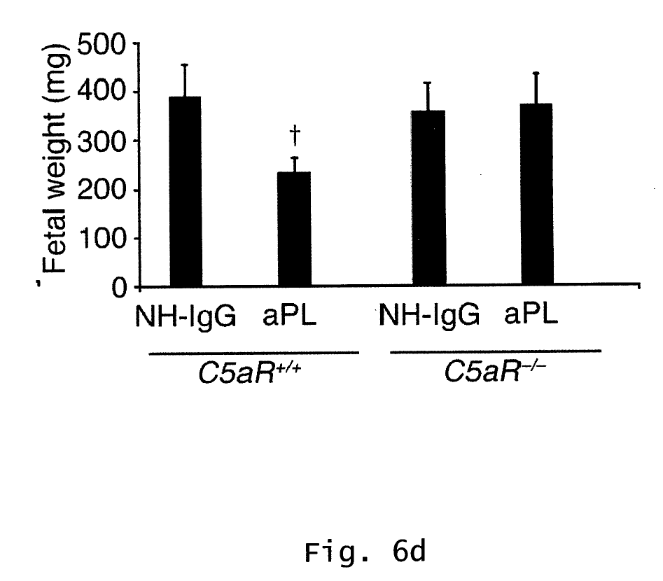

Two complement effector pathways are initiated by cleavage of C5: C5a, a potent anaphylatoxin and cell activator, and C5b, which leads to formation of the C5b-9 membrane attack complex (MAC). We used two methods to distinguish the role of C5a and the C5a receptor (C5aR) from that of MAC seeded by C5b. First, we treated pregnant mice that had received aPL-IgG with a highly specific peptide antagonist of C5aR, AcPhe[L-ornithine-Pro-D-cyclohexylalanine-Trp-Arg], which possesses potent in vivo anti-inflammatory activities in murine models of endotoxic shock, renal ischemia-reperfusion injury and the Arthus reaction (Finch et al., J. Med. Chem. 1999;42:1965-1974; Mastellos et al., J. Immunol. 2001;166:2479-2486; Strachan et al., J. Immunol. 2000;164:6560-6565; Arumugam et al., Kidney Int. 2003;63:134-142). Administration of C5aR antagonist peptide prevented aPL-antibody induced pregnancy loss and growth restriction, but had no effect on either frequency of fetal resorption or fetal size in the absence of aPL antibodies (FIGS. 6a-d). Fetal protection conferred by the C5aR antagonist was comparable to that seen with anti-C5 mAb and in mice lacking C5 (FIGS. 4 and 5), suggesting that downstream pathogenic effects are mediated predominantly by C5a-C5aR interactions. Immunohistological analysis of decidual tissue from mice treated with aPL-IgG and C5aR antagonist peptide yielded results similar to those in C5^{-/-} mice. There was minimal C3 deposition surrounding normal appearing fetuses and no evidence of inflammation.

As a second approach to test the hypothesis that C5a-C5aR interactions mediate aPL-induced pregnancy complications, we performed studies in mice deficient in C5aR. In the background strain, C5aR^{+/+}, there was a 5-fold increase in the frequency of fetal resorption after treatment with aPL-IgG (FIG. 6c), while, as predicted by experiments with the C5aR antagonist peptide, aPL-IgG did not increase the frequency of fetal resorption in C5aR^{-/-} mice (FIG. 6c). The protective effects of the total absence of C5aR were also observed when fetal weights were examined (FIG. 6d). Taken together, our experiments with C5aR^{-/-} mice and C5aR antagonist peptide identify the C5a-C5aR interaction as a critical effector of aPL antibody-induced injury.

### Depletion of Neutrophils Protects Against aPL-induced Pregnancy Complications

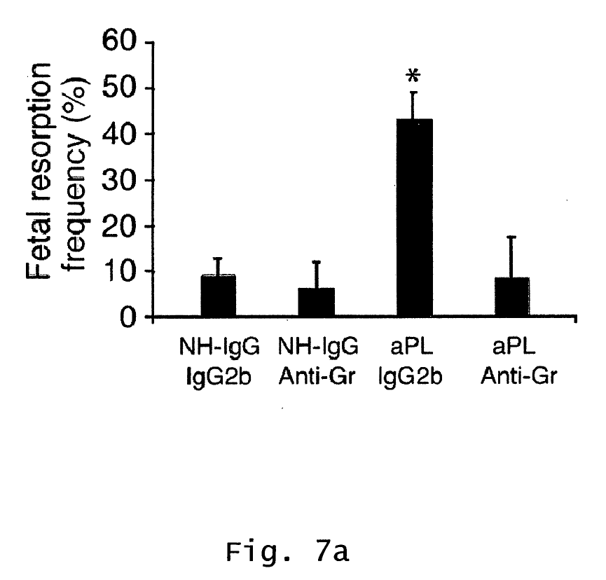

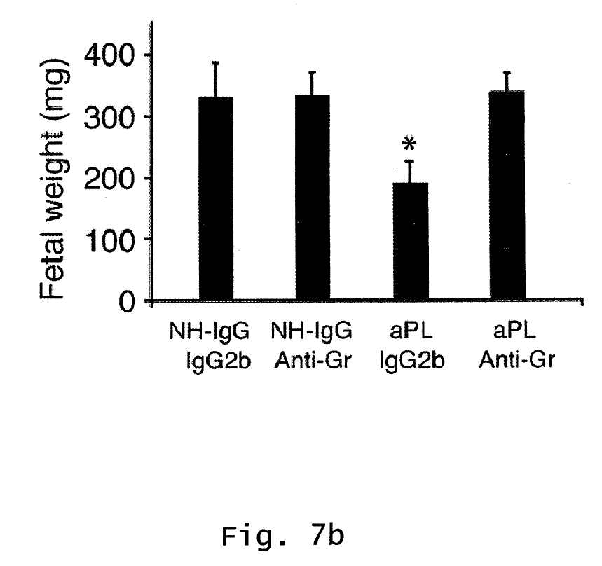

C5a is a potent chemotactic factor and activator of neutrophils. Since we observed neutrophil infiltration at sites of fetal resorption and demonstrated that the C5a-C5aR interaction is necessary for aPL antibody-induced pregnancy loss, we hypothesized that neutrophils were the critical cellular effectors of fetal damage. Indeed, neutrophils have been implicated as effectors in pathogenic antibody-induced arthritis and in antibody-independent murine models of pregnancy loss (Grant et al., J. Exp. Med. 2002;196:1461-1471; Clark et al., J. Immunol. 1998;160:545-549). To examine the relative importance of these cells in aPL antibody-initiated damage, we depleted neutrophils on day 7 of pregnancy by treating mice with rat anti-mouse granulocyte mAb RB6-8C5 (anti-Gr); IgG2b antibody served as the isotype control. In the absence of neutrophils, treatment with aPL-IgG did not cause pregnancy loss or growth restriction, nor were there inflammatory infiltrates within the deciduas (FIGS. 7a-b). Further, without neutrophil infiltration, there was less C3 deposition. These findings are similar to the limited C3 deposition we observed in C5^{-/-} mice and in C5aR blockade.

To exclude the possibility that neutrophil depletion due to treatment with anti-Gr antibody caused complement consumption, we measured circulating functional C3 levels using a zymosan activation assay before and after treatment with anti-Gr antibody (n=4 mice) (Foley et al., Eur. J. Immunol. 1993;23:1381-1384). Functional C3 measured at 9 time points from 6 to 32 hrs after anti-Gr treatment ranged from 95% to 107% of pre-treatment levels. At no point was there evidence for a significant decrease in C3, indicating protection against aPL antibody-induced pregnancy loss afforded by anti-Gr antibody treatment is not due to complement consumption by IgG-opsonized neutrophils. Rather, our results are consistent with the conclusion that neutrophils contribute directly to fetal injury. Thus, while among its many effects C5a can activate platelets, endothelial cells, and mononuclear phagocytes, it appears that C5a-mediated recruitment (and likely activation) of neutrophils in the placenta is critical for the development of pregnancy loss and fetal damage.

### Alternative Pathway of Complement Activation Contributes to aPL Antibody-induced Fetal Loss

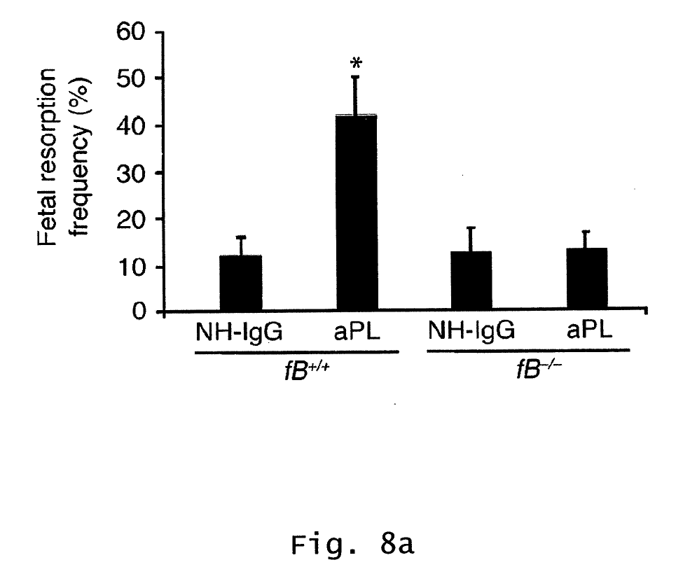

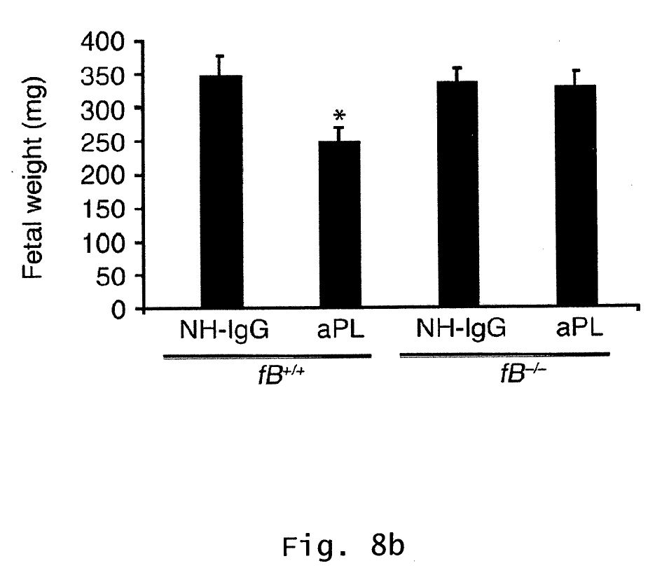

In the absence of neutrophil infiltration in decidual tissue, whether as a consequence of blockade of C5a-C5aR interactions or neutrophil depletion, we observed limited activation of C3 and improved pregnancy outcomes. It has been suggested that neutrophils promote complement deposition by causing tissue damage which triggers complement activation, and by secreting C3 and/or properdin at sites of inflammation to amplify complement activation via the alternative pathway (Schwaeble et al., Immunol. Today 1999;20:17-21; Wirthmueller et al., J. Immunol. 1997;158:4444-4451). Given the importance of neutrophils in our model of APS and their potential role as activators of the alternative pathway, we examined the contribution of this pathway of complement activation in aPL antibody-induced pregnancy loss by performing studies in mice deficient in factor B (fB). We found that fB^{-/-} mice were protected from fetal resorption and growth restriction caused by aPL antibodies. The frequency of pregnancy loss in fB^{-/-} mice treated with aPL-IgG was comparable to that observed in mice treated with control IgG (FIG. 8a). In contrast, fetal wastage and growth restriction was evident in fB^{+/+} (background strain) mice treated with aPL-IgG (FIG. 8a and b). Immunohistochemical analyses showed substantially less C3 deposition in decidual tissues and embryos from fB^{-/-} mice treated aPL antibodies than in fB^{+/+} mice. We confirmed the immunohistochemistry results with analysis of lysates from decidual cells by Western blotting probed with anti-mouse C3. Taken together, these findings indicate that the alternative pathway is an amplifier of complement activation triggered by aPL antibodies targeted to the deciduas.

### Discussion

We have shown, in a murine model of APS induced by passive transfer of human aPL antibodies, that complement activation plays an essential and causative role in fetal loss and tissue injury, and, in contrast to other models of antibody-mediated disease, that activating FcγR are not required for aPL antibody-induced effects. Specifically, we have identified the pro-inflammatory sequelae of C5a-C5aR interactions and the recruitment of neutrophils as the critical intermediates linking pathogenic aPL antibodies to fetal damage. Our conclusions are based on the fetal protective effects of C5aR deficiency and C5aR antagonist peptide, on similar findings with anti-C5 mAb and in C5^{-/-} mice, where C5a generation is prevented, and on the effects of neutrophil depletion.

Our observations that C4^{-/-} mice are protected from aPL antibody-induced pregnancy loss and that F(ab)′₂ fragments of aPL-IgG do not cause fetal injury indicate that the classical pathway is the initiator of complement activation and is required for tissue damage. Generation of C5a, through activation of the classical complement pathway, amplifies the effects of aPL antibodies targeted to the placenta. C5a attracts and activates neutrophils, monocytes, and mast cells, and stimulates the release of inflammatory mediators, including reactive oxidants, proteolytic enzymes, chemokines and cytokines, as well as complement components. Proteases secreted by inflammatory cells, particularly neutrophils, can also increase C5a generation by directly cleaving C5 (Huber-Lang et al., Am. J. Pathol. 2002;161:1849-1859), leading to autocrine and paracrine stimulation and further recruitment of leukocytes.

One of our most striking findings is that C3 deposition in decidual tissue of aPL antibody-treated mice is diminished in the absence of C5 activation and C5a release. We observed this phenomenon in mice treated anti-C5 mAb antibody, mice lacking C5 or C5aR, and mice treated with C5aR antagonist peptide. While decreased C3 deposition as a consequence of C5 activation blockade may appear counterintuitive because C3 activation precedes C5 activation, this finding can be explained by the coincident inhibition of neutrophil infiltration. In each setting where C5 activation was blocked, neutrophils were absent from decidual tissues, and in neutrophil-depleted mice, C3 deposition was substantially decreased. Thus, C3 activation and deposition do not appear to be solely dependent on complement components, as these are ample in the plasma and extracellular fluid; rather, in the absence of neutrophils there is limited amplification of the cascade and cleavage of C3.

Because apoptotic and necrotic cells activate alternative and classical pathways, neutrophil-induced cell damage can in-and-of-itself increase C3 deposition in decidual tissues (Mevorach et al., J. Exp. Med. 1998;188:2313-2320). In addition, neutrophils can enhance complement activation by releasing complement components, including C3 and properdin, a critical positive regulator of the alternative pathway. Properdin fimctions by stabilizing the interaction of factor B with spontaneously generated initial C3(H₂O) and the formation of the C3 convertase C3bBb (Schwaeble et al., Immunol. Today, 1999;20:17-21; Wirthmueller et al., J. Immunol. 1997;158:4444-4451). Such positive regulatory activity permits properdin to significantly enhance alternative pathway C3 activation resulting either from initiation of this pathway directly by C3(H₂O) formation, or indirectly through the amplification loop, which utilizes C3b generated from the classical pathway C3 convertase C4b2a. Thus, properdin and C3 secretion by neutrophils can accelerate alternative pathway activation at sites of leukocyte infiltration, enhancing C3 activation and deposition (Schwaeble et al., Immunol. Today 1999;20:17-21). Our results suggest that initial C3 deposition, catalyzed by classical pathway activation, leads to C5a generation, attracting neutrophils and potentially triggering properdin release. Furthermore, the experiments in the fB^{-/-} mice support the possibility that properdin and the alternative pathway generate most C3 at sites of injury and initiate a positive feedback loop that generates additional C5a. Briefly, according to this model, APL Ab's are preferentially targeted to the placenta where they activate complement via the classical pathway leading to the generation of potent anaphylatoxins and mediators of effector cell activation, particularly C5a. C5a attracts and activates neutrophils, monocytes, and platelets and stimulates the release of inflammatory mediators, including reactive oxidants, proteolytic enzymes, chemokines, cytokines, and complement factors C3 and properdin. Secretion of C3 and properdin by neutrophils, as well as the presence of apoptotic and necrotic decidual tissue, can accelerate alternative pathway activation (dashed line), creating a proinflammatory amplification loop at sites of leukocyte infiltration that enhances C3 activation and deposition and generates additional C5a. This results in further influx of neutrophils, inflammation within the placenta, and, ultimately, fetal injury. Depending on the extend of the damage, either death in utero or fetal growth restriction ensues. PMN, neutrophil; Mθ, monocyte/macrophage. Our findings are novel in that they link alternative pathway activation to neutrophil infiltration, and raise the possibility that infiltrating cells regulate local complement activation.

That blockade of C5 or C5aR is effective in preventing fetal injury in APS has important therapeutic implications. Blocking the complement cascade at C5 inhibits mediators and effectors of tissue injury while preserving the complement-derived immunoprotective functions of C3. Complement inhibitors are now being tested in patients with inflammatory, ischemic and autoimmune diseases. Identifying complement-related markers that predict high risk for fetal loss will allow us to translate insights about the mechanisms of complement-mediated disease to interventions that can prevent, arrest, or modify the deleterious effects of aPL antibodies.

### Example 3

This Example describes the ability of a monoclonal antibody, mAb A1379, to inhibit the alternative complement pathway in a model of aPL mediated fetal loss. As reported herein, mice deficient in factor B are greatly protected from fetal loss, indicating that an exogenous inhibitor of the alternative pathway would be an effective therapeutic agent for preventing fetal loss.

### Materials and Methods

Mice. Targeted deletion of mouse factor B was accomplished as previously described (Matsumoto et al., Proc. Natl. Acad. Sci. USA 1997;94:8720). The factor B deficient mice were created with Sv129 strain embryonic stem cells and were then crossed with C57BL/6 mice prior to expansion of the colony at Fl. C57/ B6J (Jackson Laboratories, Bar Harbor, ME) mice were used for pharmacokinetic experiments or for the collection of normal mouse serum. Adult BALB/c mice (2-3 months old) were purchased from Taconic Farms (Germantown, New York, USA) and were used in experiments involving injection of antiphospholipid antibody. Procedures that involved mice were approved by the local Committee on Animal Use in Research and Education and were conducted in strict accordance with guidelines for the care and use of laboratory research animals established by the NIH.

Production and purficdtion of mfB-SCR2,3-Ig. A plasmid encoding the second and third short consensus repeats (SCR) of mouse factor B linked to the hinge, CH2, and CH3 domains of mouse IgG1 isotype was constructed using the identical strategy as previously reported for the five extra-cellular SCRs of Crry to produce Crry-Ig (Quigg et al., J. Immunol. 1998;160:4553). The second and third SCR domains of mouse factor B were chosen because they are part of the deleted segment of the factor B gene in the fB−/− mice used in these studies (Matsumoto et al., Proc. Natl. Acad. Sci. USA 1997;94:8720), and previous mutagenesis studies (Hourcade et al., J. Biol. Chem. 1995;270:19716) had implicated these regions (and not SCR 1) as critical to factor B hemolytic function. The resulting plasmid p118-mfBSCR2,3-Ig was transfected into the NS/0 nonsecreting hybridoma cell line and clones producing mfB-SCR2,3-Ig were selected by limiting dilution, G418 drug resistance, and ELISA as described (Quigg et al., J. Immunol. 1998;160:4553). The highest producing clone was chosen for further analysis and expanded into T175 flasks with Iscove's modified Dulbecco's medium (Invitrogen, Carlsbad, Mass.) supplemented with 10% FC5, nonessential amino acids, sodium pyruvate, and pen/strep/glutamine.

To purify mfB-SCR2,3-Ig, approximately two liters of tissue culture supernatant were harvested, filtered through a 0.45-μm filter, and concentrated 20 fold using a pressure cell equipped with a YM10 spiral wound ultrafiltration cartridge (Amicon, Bedford, Mass.) at 4° C. The concentrate was dialyzed against PBS, pH 7.4 and then loaded onto a 15×2 cm affinity column that was pre-packed with anti-mouse IgG1-agarose (Sigma) and equilibrated with PBS. After washing the column with PBS the column was stripped with 5M LiCl₂ to elute the affinity purified protein. The LiCl₂ eluted protein was concentrated using a Centricon centrifuigal filtration device (Amicon) and dialyzed against PBS. The recovered protein was analyzed by SDS-PAGE on a Novex® tris-glycine gel (Invitrogen) and stained with Coomassie blue. The highly purified band of approximately 110 kDa (FIG. 1C) was subjected to amino-terminal sequence analysis to assure proper signal peptide cleavage of the CD5 leader peptide. The result demonstrated the sequence of IRCPRPQDFE (SEQ ID NO: 3) which is the authentic mfB-SCR2,3-Ig protein.

Purification of mouse factor B. Mouse complement factor B was purified from normal mouse serum by affinity purification. The affinity column was created by binding goat anti-human properdin factor B (Diasorin, Stillwater, MN) to CNBr-Activated Separose (Amersham, Arlington Heights, IL) according to the manufacturer's instructions. C57/ B6J mice were bled by cardiac puncture, and the blood was collected into syringes containing 50 μl of 500 mM EDTA in order to prevent alternative pathway activation. The blood was centrifuged at 2000 rpm for 15 minutes and the plasma was collected. The plasma was then diluted 1:1 with buffer (EACA 50 mM, EDTA 10 mM, benzamidine 2 mM in PBS, pH 7.4) and passed through a 0.22 μm filter (GE Water Technologies). The plasma was added to the affinity column and the column was washed with 10 column volumes of buffer. The factor B was eluted using 5 M LiCl₂ and dialyzed overnight against PBS. The purity of the factor B was then analyzed by electrophoresis on a 10% Tris-Glycine gel and stained with Coomassie.

Development of inhibitory mabs targeting factor B. Factor B deficient mice were immunized with 125 μg of the recombinant factor B-Ig fusion protein emulsified with incomplete Freund's adjuvant and then boosted four times at three week intervals. The mice were screened for the development of inhibitory antibodies to factor B by testing their sera in an ELISA using mouse factor B coated plates and an in vitro assay of alternative complement pathway inhibition (described below). One day after the last injection, spleen cells from a mouse identified as having a robust inhibitory immune response towards factor B were fused to a myeloma cell line in the University of Colorado Monoclonal Antibody Center. Candidate hybridomas were cloned by limiting dilution, and clones capable of recognizing mouse factor B by ELISA and inhibiting alternative pathway activity were identified. One of the hybridomas, designated 1379, was identified as an effective inhibitor of the alternative pathway. 1379 was purified from tissue culture supernatant with a Protein-G Sepharose column (Pharacia, Uppsala, Sweden). LPS was removed from the purified mAb using polymyxin (Sigma-Aldrich, St. Louis, Mo.). The Limulus Amebocyte Lysate Assay (BioWhittaker, Inc., Walkersville, Md.) was used according to the manufacturers instructions to verify that the mab had LPS levels below 1 EU/mg of mAb. The purity of the mAb was then analyzed by electrophoresis on a 10% Tris-Glycine gel and stained with Coomassie.

ELISA analysis of anti-factor B antibody levels. Mice were screened for an immune response to the immunizations by testing their sera in an enzyme linked immunosorbent assay (ELISA) against purified mouse factor B. Ninety-six well ELISA plates (Costar, Corning, N.Y.) were coated with 125 ng of purified factor B in coating buffer (15 mM Na₂CO₃, 35 mM Na₂HCO₃) and stored overnight at 4° C. The plates were then washed with 200 μl of PBS. Non-specific binding was blocked by incubating the plates with 200 μl of 5% BSA (Sigma) in PBS. The plates were washed two times with 200 μl of PBS with 0.1% Tween 20 (Sigma), then incubated with diluted serum for one hour. Samples were diluted 1:100 in PBS with 0.1% Tween-20 and 0.1% BSA, then the samples were further serially diluted 1:1 seven times. The plates were then washed two times and incubated with 50 μl of peroxidase conjugated goat anti-mouse IgG (Cappel, Durham, NC). The plates were next washed four times and incubated with 100 μl of ABTS containing 1:1000 30% H₂O₂ (Sigma), and absorbance at 405 nm was read with a microplate reader (Biorad, Richmond, Calif.).

Assays of alternative complement pathway inhibition. Sera with detectable titers of anti-factor B Ab were screened for the ability to inhibit the alternative pathway. This was performed using an in vitro analysis of C3 deposition on zymosan A particles (Sigma) (Quigg et al., J. Immunol. 1998;160:4553). Fifty mg of zymosan particles in 10 ml of 0.15 M NaCl were boiled for 60 minutes, then washed twice in PBS. Sera was assayed by mixing 1×10⁷ zymosan particles in a reaction mix with a final concentration of 10 mM EGTA and 5 mM MgCl₂. Ten microliters of sera from unmanipulated C57/ B6 mice were added as a source of complement. Assays of inhibition were conducted with up to 70 μl of sera from immunized mice (to screen for the generation of inhibitory antibodies) or with purified antibody titrated from 0.125 μg to 4 μg per reaction. Samples were brought up to 100 μl final volume with PBS and were incubated at 370 C for 30 minutes. The zymosan particles were washed twice with cold PBS, 1% fetal bovine serum, and were then incubated with FITC-conjugated goat anti-mouse C3 (Cappel, Durham, NC) for one hour on ice. The samples were again washed twice, were resuspended in 0.5 ml of PBS, 1% fetal bovine serum, and were then analyzed by flow cytometry. Percent inhibition was calculated using the formula:

$$100 \times [ 1 - \frac{( sample mean channel fluorescence - background ( noserum )}{( positive control mean channel fluorescence - background} ] \qquad ( I )$$

Fab fragments of the 1379 clone were also tested for the ability to inhibit the alternative pathway using the zymosan assay. Fab fragments were generated by incubating purified antibody with papain-agarose (ICN Biomedicals, Aurora, Ohio) according to the manufacturer's instructions. Fc fragments and undigested IgG were then removed by applying the digested antibody to a protein G column. The Fab fragments were collected in the flow through, and the Fc fragments and undigested IgG were subsequently eluted with 0.1 M glycine-HCl, pH 2.8. One μg of the Fab was used in the zymosan reaction. The polyclonal anti-mouse C3 antibody used in the zymosan assay was found to have cross reactivity with multiple species. This assay was therefore used to test inhibition by the 1379 clone of the alternative pathway in those species. Titration of the inhibitory antibody was conducted as described above.

As another assay of the ability of the 1379 clone to inhibit the alternative complement pathway, we tested the ability of this antibody to inhibit lysis of unsensitized rabbit erythrocytes by human serum. Whole rabbit blood was purchased (Colorado Serum Company, Denver, Colo.), and was mixed 1:1 with a buffer solution composed of 2.05% dextrose, 0.8% sodium citrate (dihydrate), 0.4% NaCl, 0.055% citric acid. Five ml of the erythrocyte solution was then mixed 1:9 with a solution of 1.1% NaCl, 0.0025% Na-5,5 diethyl barbiturate, pH 7.35, 8 mM EGTA, 2 mM MgCl₂. The mixture was incubated at 37° C. for several minutes then centrifuged at 1000 ×g for 10 minutes at 4° C. The erythrocytes were washed three more times before being resuspended in 40 ml of the same solution. Fifty μl of this suspension was added to human serum (5 to 100 μl), and buffer solution was added to bring the final volume up to 150 μl. Erythrocytes in buffer without serum were used as a negative control, and erythrocytes added to 100 μl of distilled water were used as positive controls (complete lysis). Samples were incubated at 37° C. for 30 minutes with occasional shaking to keep the cells in suspension. The reactions were stopped by adding 1.5 ml of cold PBS and the samples were spun at 1000 ×g for five minutes. The optical density of each supernatant was read at 415 nm using a spectrophotometer (Biorad). Ten μl of serum were found to cause complete lysis of the erythrocytes. The same reaction was then carried out using 10 μl of the serum and increasing concentrations of the mAb 1379 (0.125 μg to 8 μg per reaction). Percent inhibition of alternative pathway activity was determined using the formula:

$$100 \times [ 1 - \frac{{OD}_{sample} - {OD}_{background}}{{OD}_{positive control} - {OD}_{background}} ] \qquad ( II )$$

In vivo pharmacokinetics of mAb 1379. Mice were pre-bled, and then were injected intraperitoneally (IP) or intravenously (IV) with 0.5 or one mg doses of mAb 1379. These doses were chosen because we estimated that they would be equimolar with factor B. Factor B was estimated to be present in the serum at approximately 200 μg/ml (or ˜2.2 μM given that factor B is a 90 kD protein). Because mAb 1379 is 150 kD and the intravascular volume of an adult mouse is approximately 3 ml, a one mg injection (6.7 μMol) should result in a circulating concentration of ˜2.2 μM. Because the mAb is divalent, we anticipated that this equimolar injection would be more than sufficient to completely inhibit the alternative pathway. The mice were bled 1, 2, 6, 24, 48, and 96 hours after the injection of the inhibitor. Sera from these timepoints were then used in the zymosan assay to assess the activity of the alternative pathway.

Mapping of the mAb A1379 epitope using factor B mutants. Because mAb 1379 recognized and blocked the activity of human factor B, a panel of 22 human factor B mutants was used to characterize the mAb A1379 binding site. The mutant designated B16/17 substitutes 136-Gly-137-Ala-138-Gly with Gln-Ser-Ser [numbering of Mole et al (Mole et al., J. Biol. Chem. 1984;259:3407)]. Mutant B23/24 substitutes 187-Ser-188-Gly-189-Thr-190-Pro-191-Ser (SEQ ID NO:4) with Asp-Gly-Glu-Thr-Ala-Val (SEQ ID NO:5). Mutant B25 substitutes 210-Thr-211-Pro-212-Gln-213-Glu-214-Val-215-Glu-216-Ala (SEQ ID NO:6) with Phe-Pro-Glu-Asp-Val-Ala-Pro (SEQ ID NO:7). The other mutants have been previously described (Hourcade et al., J. Biol. Chem. 1995;270:19716). Each factor B mutant was constructed by substitution of several amino acids in SCRs 2 or 3 of the Ba region using site-directed mutagenesis. Mutant proteins were produced by transient expression in Cos or 293T cells and quantitated by ELISA (Hourcade et al., J. Biol. Chem. 1995;270:19716). To measure mAb:factor B recognition, microtiter wells were treated overnight at 4 C with 1:2000 diluted goat anti-human factor B polyclonal antibody (Incstar, Minneapolis, Minn.). Wells were then blocked for 1 hr at 37° C. with 1% BSA, 0.1% Tween 20 in PBS and stored at 4° C. until use. Prepared wells were washed and treated for 1 hr at 37 C with 100 ng/ml wild type native factor B, wild type recombinant factor B, mutant factor B, or negative control cell supernatant (Hourcade et al., J. Biol. Chem. 1995;270:19716). Wells were extensively washed and then incubated for 1 hr at 37 C with either 100 ng/ml A1379 or 50 ng/ml anti-Bb mAb (a reagent that detects wild type factor B and all of the mutant forms of factor B described in this study; Quidel, San Diego, Calif.) as a positive control. Following washing, peroxidase-conjugated donkey anti-mouse IgG (1:5000; Jackson Immunoresearch Laboratories, West Grove, Pa.) was added and OD₄₁₄ was measured by microplate reader. All standards, samples and controls were assayed in duplicate. Recognition of factor B proteins by mAb 1379 was normalized to that of the anti-Bb mAb and expressed as the ratio: (OD 1379 reacted with mutant/OD 1379 reacted with fB wt)/(OD anti-Bb reacted with mutant/OD anti-Bb reacted with fB wt). The values given in the figure represent averages of 2 separate experiments.

Factor D-dependent assembly of C3bBbP-Mg2+ complexes measured by ELISA. Wild type human factor B (400 ng/ml) in PB supplemented with 2 mM NiCl₂, 25 mM NaCl, 4% BSA, and 0.05% Tween 20 was incubated for 30 min at 25 C alone or with mAb 1379 (concentrations described in the Figure Legend). Human factor D (25 ng/ml) was then added and the final mixtures applied to C3b-coated wells for 1 hr at 37 C. Wells were extensively washed and the remaining C3bBbNi (2+) complexes detected by ELISA using goat anti-human factor B polyclonal antibody followed by peroxidase-conjugated rabbit anti-goat polyclonal antibody (Hourcade et al., J. Biol. Chem. 1995;270:19716; Hourcade et al., J. Biol. Chem. 1998;273:25996). Factor B, C3b and factor D proteins were purchased from Advanced Research Technologies.

Modeling of the epitope recognized by mAb 1379. A three dimensional model of factor B was constructed using the spatial coordinates of factor H SCR 16 (Protein Data Bank accession number 1HCC (Norman et al., J. Mol. Biol. 1991;219:717)) and the automated program MODELLER (Sali et al., J. Mol. Biol. 1993;234:779) using the INSIGHTII software platform (Accelrys, San Diego, Calif.). The amino acid sequences of human factor B SCR 3 (Mole et al., J. Biol. Chem. 1984;259:3407) and human factor H SCR 16 (Ripoche et al., Biochem J. 1988;249:593) were aligned with the INSIGHTII HOMOLOGY module (Accelerys).

Preparation of aPL and other Abs. Human IgG containing aPL Ab's (aPL-IgG) were obtained from three patients with APS characterized by high-titer aPL Ab's (>140 GPL units), thromboses, and/or pregnancy losses as previously described (Girardi et al., J. Clin. Invest. 2003;112:1644). IgG was purified by affinity chromatography using protein G-Sepharose chromatography columns (Amersham Pharmacia Biotech, Piscataway, New Jersey, USA). Control human IgG from healthy non-autoimmune individuals was purified by an identical method. Polyclonal mouse IgG control was obtained from Jackson ImmunoResearch Laboratories. All IgG samples were treated to deplete endotoxin with Centriprep ultrafiltration devices (Millipore) and determined to be free of endotoxin contamination by the limulus amebocyte lysate assay to a sensitivity of 0.06 EU/ml (Associates of Cape Cod).

Murine pregnancy loss model. Females were mated with previously isolated males. The presence of a vaginal plug was defined as day 0 of pregnancy. On days 8 and 12 of pregnancy, mice were treated with intraperitoneal injections of aPL-IgG (10 mg) or NH-IgG (10 mg). To inhibit factor B, mice were injected on days 7 through 13 of pregnancy with mAb 1379 (2 mg intraperitoneally) or with murine IgG as a control. Mice were sacrificed on day 15 of pregnancy, uteri were dissected, fetuses and placentas were weighed, and fetal resorption rates were calculated (number of resorptions per total number of formed fetuses and resorptions). Resorption sites are easily identified and result from loss of a previously viable fetus. Serum C3adesArg levels were measured by an ELISA according to the manufacturer's instructions (Cedarlane Laboratories Limited, Ontario, Canada).

Immunohistochemistry. Deciduas were removed from mice on day 8 of pregnancy 60 minutes after administration of aPL-IgG, frozen in OCT compound, and cut into 10-μm sections. After quenching endogenous peroxidase with 1% H₂O₂ in methanol and blocking non-specific binding sites with normal goat serum (Cappel), sections were incubated with goat anti-mouse C3 or goat antihuman IgG (Cappel) followed by anti-goat IgG conjugated to HRP (Sigma-Aldrich). Sections were counterstained with hematoxylin.

Statistical analyses. Data are expressed as mean± SEM. Multiple group comparisons were performed using ANOVA with posttest according to Newman-Keuls. A Student's t test was used to compare fetal resorption rates and fetal weights between groups. A Mann-Whitney U test was used to compare values for semiquantitative scoring of immunohistochemistry. P values of less than 0.05 were used to reject the null hypothesis.

### Results

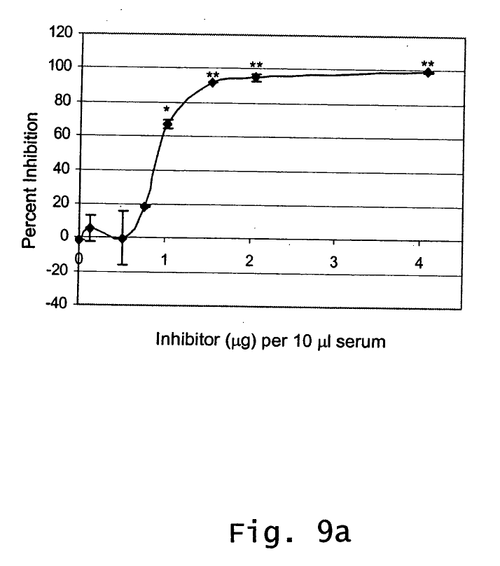

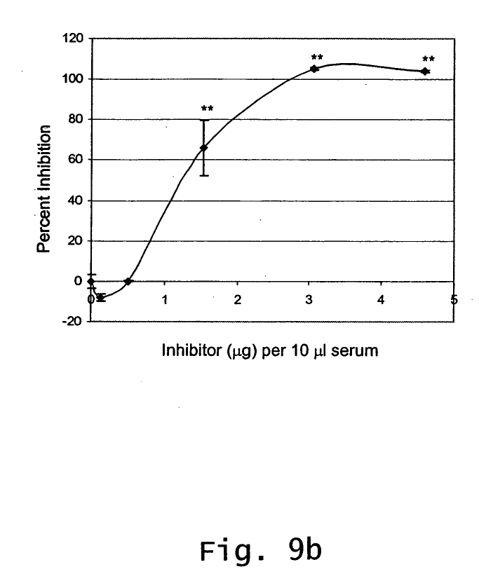

Generation of inhibitory mabs to the Ba portion offactor B. Monoclonal antibodies to mouse factor B were generated as described in the Methods section. Sera from immunized mice were assayed for the presence of anti-factor B antibodies. After the creation of hybridomas from the splenic B cells of this mouse, supernatants from the clones were tested using two in vitro assays of alternative pathway activity (FIG. 9). One clone, designated 1379, was chosen for further characterization due to the fact that the hybridoma was found to be rapidly growing, the antibody was of the mouse IgG₁ subclass (non-complement activating), and its supernatant was found to be a potent inhibitor of the alternative complement pathway.

The addition of 1.5 μg of mAb to a zymosan reaction containing 10 μl of serum caused nearly complete inhibition of the alternative pathway (FIG. 9A). The anti-factor B mAb and factor B are approximately equimolar at this concentration (Assuming that factor B is present at 200 μg/ml and has a molecular weight of 90,000 kD, there are 0.022 nMol in 10 μl of serum, and 1.5 μg of antibody with a molecular weight of 150,000 kD equals approximately 0.01 nMol). In the rabbit erythrocyte lysis assay, full inhibition was achieved with 3 μg of antibody per 10 μl of human serum in the reaction. Inhibition of the alternative pathway was next tested using Fab fragments made from the 1379 clone. When 12.5 μg of Fab from the 1379 clone was used, complete inhibition of alternative pathway activity was seen by this assay.

The ability of mAb 1379 to inhibit alternative pathway activity in sera from multiple different mammalian species was also tested using the zymosan assay. The 1379 mAb was able to fully inhibit alternative pathway activation when 8 μg of mAb were added to a reaction containing 10 μl of sera from most of the species tested (Table 1). The antibody fully inhibited alternative pathway activity in serum from mice, rats, humans, and several species of 10 monkeys. It did not, however, demonstrate any inhibitory activity towards serum from dogs or guinea pigs.

TABLE 1
Species tested for inhibition with the mAb 1379. The anti-factor B
mAb 1379 was tested for its ability to inhibit serum from 10
different species in the Zymosan assay (described in Methods
section). The antibody completely inhibited alternative pathway
activity in eight of the ten species tested, and there was no
measurable inhibition in the final two.
Species in which the alternative pathway
is fully inhibited by mAb 1379

Mouse
Human
Rat
Baboon
Rhesus
Cyno Monkey
Pig
Horse

Species in which the alternative pathway is not inhibited by mAb 1379

Dog
Guinea Pig

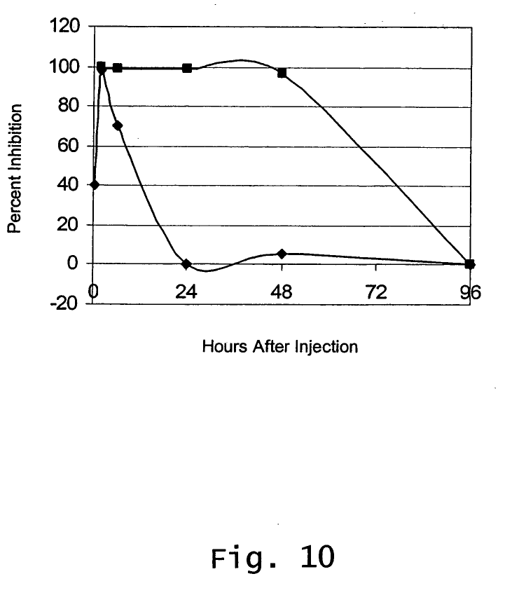

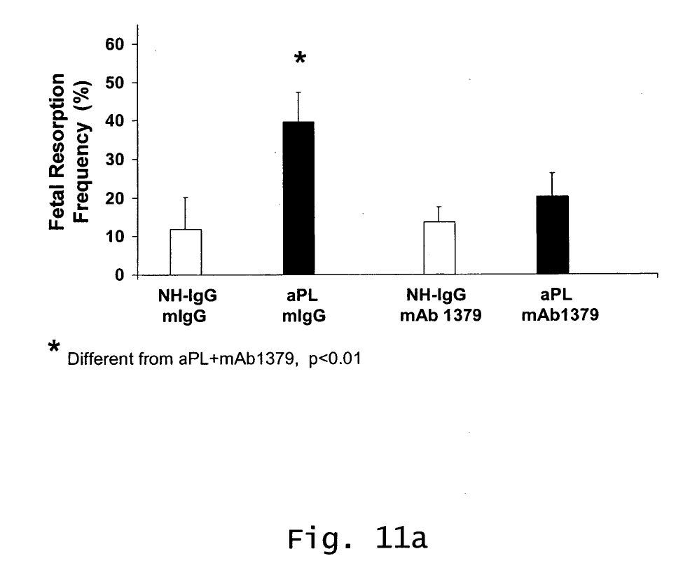

Pharmacokinetics of mAb 1379. Mice were tested for inhibition of the alternative pathway at various times after a single injection of the inhibitory antibody. Injection with 0.5 mg of antibody led to full inhibition within one hour when injected IV and within two 20 hours when injected IP (FIG. 10). Mice receiving a 0.5 mg injection IP retained full inhibition of the alternative pathway at 24 hours and those receiving a one mg injection retained full inhibition up to 48 hours after the injection.

1379 binds an epitope in the SCR3 region of Ba. The ability of the 1379 mAb to bind to a panel of factor B mutants was performed as previously described (Hourcade et al., J. Biol. Chem. 1995;270:19716) in order to characterize the mAb binding site. From the 22 different mutants tested, 1379 had virtually no binding to the B17 and B23 mutants and retained less than 20% of its binding capability to the B18 mutant. The factor B mutants that were not recognized by mAb 1379 each have mutations within the SCR3 region. Mutant B17 substitutes 139-Tyr-140-Cys-141-Ser with His-Cys-Pro; mutant B23 substitutes 182-Glu-183-Gly-184-Gly-185-Ser with Gly-Asn-Gly-Val. B17 and B23 proteins are 54 and 22% active, respectively, in a factor B-dependent hemolysis assay (Hourcade et al., J. Biol. Chem. 1995;270:19716). The regions containing these two mutations make up a conserved surface on the factor B protein.

1379 prevents assembly of the C3bBb convertase. To determine whether mAb 1379 interferes with factor D dependent formation of C3bBb (the alternative pathway C3 convertase) we examined whether the mAb inhibits formation of C3bBb in an ELISA assay as previously described (Hourcade et al., Immunopharmacology 1999;42:167). Purified factor B was added to a microtiter well containing C3b and factor D, and formation of the C3bBb complex was detected with an antibody to factor B. The addition of mAb 1379 prevented formation of the C3bBb convertase in this assay.

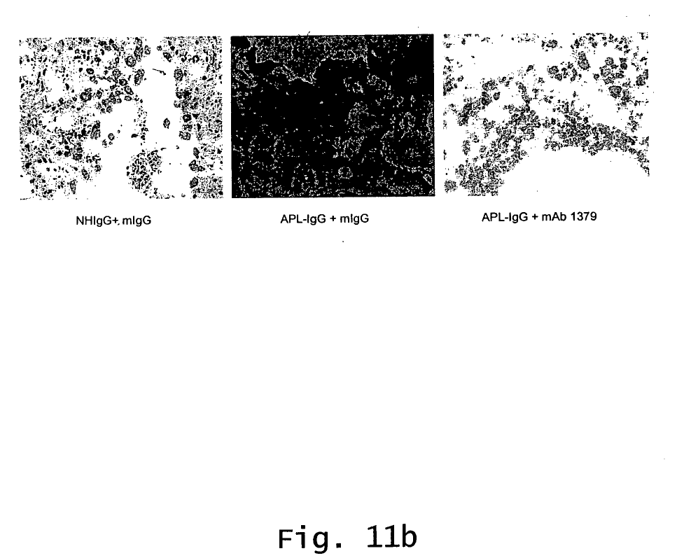

Treatment with 1379 protects micefrom aPL Ab-induced pregnancy loss. Mice deficient in factor B are protected from aPL Ab-induced fetal injury (Girardi et al., J. Clin. Invest. 2003;112:1644). Therefore, we tested whether treatment with 1379 would similarly protect mice from the fetal resorption typically seen in mice treated with aPL (Girardi et al., J. Clin. Invest. 2003;1 12:1644). Treatment with aPL-IgG caused a significant increase in the frequency of fetal resorption (39.5±7.8% compared to 11.9±8.2% in the group receiving NH-IgG), while simultaneous treatment with mAb 1379 prevented aPL antibody-induced pregnancy loss (20.3±6%) (FIG. 11A). Treatment with mAb 1379 had no effect on the fetal resorption frequency in mice that received NH-IgG (FIG. 11B).

Staining of decidual tissue with antibodies against complement component C3 revealed extensive complement deposition in the mice treated with aPL-IgG. Increased neutrophil infiltration was observed in deciduas from aPL-IgG treated mice. Treatment with mAb 1379 completely prevented aPL-IgG induced C3 deposition and neutrophil infiltration on the deciduas.

Plasma C3adesArg, a product of the cleavage of C3a, was determined as a measure of systemic complement activation. aPL-IgG treated mice showed increased C3adesArg plasma levels compared to NH-IgG treated mice (1212.1±101.7 ng/ml vs. 78.3.1±32.5 ng/ml at day 8, P<0.001) (Table II). aPL-IgG treated mice that received mAb 1379 did not show increased C3adesArg plasma levels (137.4±24.5 ng/ml, P<0.001 compared to treatment with aPL-IgG alone) (Table II). These results show that mAb 1379 can inhibit complement activation and inflammation associated with aPL-IgG treatment.

TABLE II
C3a levels in the sera from mice treated with aPL-Ab.

| Treatment | Plasma C3adesArg (ng/ml) |
|---|---|
| Control (n = 5) | 78.3 ± 32.5 |
| NH-IgG (n = 4) | 94.3 ± 25.8 |
| aPL day 8 (n = 6) | 1212.1 ± 101.7*^{#} |
| aPL day 12 (n = 5) | 940.5 ± 32.6*^{#} |
| aPL day 15(n = 6) | 540.6 ± 121.3*^{#} |
| aPL + 1379 day 8 (n = 6) | 137.4 ± 24.5** |
| aPL + 1379 day 12 (n = 5) | 147.1 ± 35.3** |
| aPL + 1379 day 15 (n = 6) | 142.5 ± 31.4** |

^{a}Control mice received no treatment.
NH-IgG indicates treatment with normal human IgG.
*Different from Control, p < 0.001,
^{#}Different from NHIgG, p < 0.001,
**Different from aPL, p < 0.001.

### Discussion

A novel mAb to mouse factor B has been generated. This antibody is a specific inhibitor of the alternative pathway of complement and leads to complete inhibition of this pathway in vitro and in vivo. A 2 mg ip injection led to complete inhibition of the alternative pathway in mice for up to 48 hours.

This antibody effectively protected mice from aPL Ab-induced fetal injury. Passive transfer of human IgG from patients with high titer of aPL-antibodies resulted in complement activation within the deciduas, inflammation and fetal injury with a resorption frequency of greater than 40%. Treatment with 1379 effectively reduced aPL-antibody induced fetal resorption frequency, decidual inflammation, and decidual complement activation when compared to mice receiving only aPL-antibodies. MAb 1379 also protected mice from spontaneous fetal loss, again with diminished complement activation in the placenta.

This study demonstrates that systemic administration of an inhibitor of the alternative pathway can reduce complement activation at local sites of antibody induced inflammation. The inhibitor was effective despite the fact that injury is initiated by IgG and despite the apparent effects of locally amplified alternative pathway activation by infiltrating neutrophils. Furthermore, administration of mAb 1379 significantly reduced the systemic levels of pro-inflammatory complement activation products released as a result of this activation. Our results highlight the usefulness of this inhibitory niAb for the study of the pathogenesis of any complement mediated disease.

Our results support that an exogenous inhibitor of the alternative pathway can successfully treat APS. In this model, treatment with 1379 significantly reduced fetal loss as a result of aPL. High intensity anticoagulation is currently the standard of care for patients with the aPL. The monitoring of the international normalized ratio (INR) in APS patients can be difficult (Moll et al., Ann. Intern. Med. 1997;127:177), and anticoagulation may be contraindicated in some patients. Treatment with a complement inhibitor provides an appealing alternative therapy for those who fail or cannot tolerate coumadin therapy. Anticoagulation also reduces, but does not eliminate, the incidence of recurrent thrombosis in patients with the APS (Khamashta et al., N. Eng. J. Med. 1995;332:993).

Our results support that exogenous inhibition of the alternative pathway can successfully treat spontaneous fetal loss. By specifically inhibiting only factor B, 1379 can have several advantages compared to inhibitors that work at other levels in the complement cascade. Given that C4−/− mice but not fB−/− mice appear more susceptible to experimental bacterial infection (Matsumoto et al., Proc. Natl. Acad. Sci. USA 1997;94:8720; Wessels et al., Proc. Natl. Acad. Sci. USA 1995;92:11490), by leaving the classical pathway intact this inhibitor poses less risk of serious infection than inhibitors that also block the classical pathway. Blockade of C3 can impair immune complex processing in autoimmune states, perhaps explaining why blockade of the alternative pathway ameliorates a mouse model of lupus nephritis Watanabe et al., J. Immunol. 2000;164:786), whereas C3 deficiency does not (Sekine et al., J. Immunol. 2001;166:6444). Inhibition of the alternative pathway also prevents generation of C3 derived ligands for the C3aR as well as complement receptors 1-4, whereas agents that target C5 or C5a do not.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

What is claimed is:

1. A method of preventing a miscarriage, which method comprises administering to a human female subject who is pregnant or planning to become pregnant an effective amount for preventing miscarriage in said female of an agent capable of inhibiting a component of the complement activation pathway.

2. The method of claim 1, wherein said component is a member of the group consisting of factor B, factor D, properdin, C2, C3, C3 convertase, C4, C5, C5 convertase, C3a, C5a, membrane attack complex (MAC), C3a receptor, C5a receptor and members of the mannan-binding protein (MBL) pathway.

3. The method of claim 1, wherein said female subject does not suffer from the antiphospholipid syndrome (APS).

4. The method of claim 1 which comprises inhibiting C3 conversion with the agent.

5. The method of claim 4, wherein said agent comprises a member of the group consisting of an antibody directed against C3, an antibody directed against C3 convertase, and a cyclic peptide inhibitor having the amino acid sequence of SEQ ID NO:1.

6. The method of claim 5, wherein said antibody comprises a member of the group consisting of a chimeric antibody, a humanized antibody, and a human antibody, and fragments thereof.

7. The method of claim 1 which comprises inhibiting C5 cleavage with the agent.

8. The method of claim 7, wherein said agent comprises a member of the group consisting of an antibody directed against C5 and an antibody directed against C5 convertase.

9. The method of claim 8, wherein said antibody comprises a member of the group consisting of a chimeric antibody, a humanized antibody, and a human antibody, and fragments thereof.

10. The method of claim 1, which comprises inhibiting C5a binding to the C5a receptor with said agent.

11. The method of claim 10, wherein the agent comprises a member of the group consisting of an antibody directed against C5a, an antibody directed against the C5a receptor, and AcPhe(L-omithine-Pro-D-cyclohexylalanine-Trp-Arg) (SEQ ID NO:2).

12. The method of claim 11, wherein said antibody comprises a member of the group consisting of a chimeric antibody, a humanized antibody, and a human antibody, and fragments thereof.

13. The method of claim 2, which comprises inhibiting a member selected from factor B and other alternative pathway derived mechanisms of activation of C3 with said agent.

14. The method of claim 13, wherein said agent is selected from an antibody directed against factor B, factor D and properdin.

15. The method of claim 11, wherein the antibody comprises a member of the group consisting of a mouse antibody, a chimeric antibody, a humanized antibody, and a human antibody, and fragments thereof.

16. The method of claim 2 wherein the agent comprises an anti-sense nucleic acid sequence capable of binding to a nucleic acid encoding said component.

17. The method of claim 1, wherein the female subject is pregnant and at risk for miscarriage.

18. The method of claim 17, wherein the subject has had at least one previous miscarriage.

19. The method of claim 18, wherein said previous miscarriage was not caused by a genetic, anatomic, endocrine, or infectious condition.

20. A method of preventing a miscarriage, which method comprises administering to a female subject who is pregnant or planning to become pregnant an effective amount for preventing miscarriage in said female of an agent capable of inhibiting a component which comprises a member selected from the group consisting of factor B, factor D and properdin.

21. A method of screening to identify an agent useful for treating or preventing miscarriage which comprises
(i) providing a pool of test agents;
(ii) determining whether any test agent from the pool inhibits the activity of at least one member selected from the group consisting of factor B, factor D, properdin, C2, C3, C3 convertase, C4, C5, C5 convertase, C3a, C5a, membrane attack complex (MAC), C3a receptor, C5a receptor and members of the mannan-binding protein (MBL) pathway, and
(iii) selecting any test agent from the pool that inhibits the activity of at least one member as an agent useful for treating or preventing miscarriage.

22. The method of claim 21, which comprises a step of selecting the pool of test agents prior to step (i).

23. The method of claim 21, wherein the determining step comprises the steps of:
(a) measuring the level of a complement split product formed downstream from the at least one member;
(b) comparing the level of the complement split product to a control value; and
(c) selecting any test agent for which the level of the complement split product is higher than the control value as an agent useful in treating or preventing miscarriage.

24. The method of claim 23, wherein the complement split product comprises a member selected from the group consisting of C3a, C3d, C4a, Bb, Ba, C4d, and SC5b-9.
